(19) United States
(12) Patent Application Publication
(10) Pub. No.: US 2017/0348387 A1
(43) Pub. Date: Dec. 7, 2017

(54) AAV-MEDIATED GENE THERAPY FOR NPHP5 LCA-CILIOPATHY

(71) Applicants: The Trustees of the University of Pennsylvania, Philadelphia, PA (US); University of Florida Research Foundation, Inc., Gainesville, FL (US)

(72) Inventors: Gustavo Aguirre, Media, PA (US); William A. Beltran, Philadelphia, PA (US); Samuel G. Jacobson, Penn Valley, PA (US); Artur V. Cideciyan, Lafayette Hill, PA (US); William W. Hauswirth, Gainesville, FL (US); Sanford L. Boye, Gainesville, FL (US)

(21) Appl. No.: 15/445,776

(22) Filed: Feb. 28, 2017

Related U.S. Application Data
(60) Provisional application No. 62/301,266, filed on Feb. 29, 2016.

(51) Int. Cl. A61K 38/17; A61B 3/12; A61B 3/14; C07K 14/005; A61B 3/00; C12N 15/86; C12N 7/00; C07K 14/47; C12Q 1/68; A61K 48/00

(52) U.S. Cl. CPC A61K 38/1709; C12Q 1/6883; A61B 3/12; A61B 3/14; A61K 48/0008; A61K 48/0058; A61B 3/0025; C12N 15/86; C12N 7/00; C07K 14/47; C07K 14/005; A61K 48/0075; C12N 2750/14143; C12N 2750/14171; C12N 2830/008; C12N 2750/14121; C12Q 2600/156

(57) ABSTRACT

Described herein are methods of preventing, arresting progression of or ameliorating vision loss and other conditions associated with Leber congenital amaurosis (LCA) in a subject. The methods include administering to said subject an effective concentration of a composition comprising a recombinant adeno-associated virus (AAV) carrying a nucleic acid sequence encoding a normal NPHP5 protein, or fragment thereof, under the control of regulatory sequences which express the NPHP5 protein in the photoreceptor cells of the subject, and a pharmaceutically acceptable carrier.

### STATEMENT OF GOVERNMENT INTEREST

This invention was made with government support under contract nos. EY-06855 and EY-017549 awarded by the National Institutes of Health/National Eye Institute. The government has certain rights in the invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “UPN-16-7749_Seq_Listing_ST25”.

### BACKGROUND OF THE INVENTION

Photoreceptors function cooperatively with the retinal pigment epithelium (RPE) to optimize photon catch and generate signals that are transmitted to higher vision centers and perceived as a visual image. Disruption of the visual process in the retinal photoreceptors can result in blindness. Genetic defects in the retina cause substantial numbers of sight-impairing disorders by a multitude of mechanisms. The photoreceptor (PR) sensory cilium connects the metabolically active inner segment (IS) to the outer segment (OS), and through this narrow isthmus traffic critical membrane and soluble proteins. The structural and functional complexity of the sensory cilium is evident from its four structural domains, and multiple domain-specific interacting proteins. Mutations in genes encoding these critical proteins cause diseases collectively termed ciliopathies that can affect the retina alone, or be syndromic with associated renal and CNS defects. The retinopathies can be either early or later onset, and are generally grouped clinically under the rubrics of Leber congenital amaurosis (LCA) and retinitis pigmentosa (RP). The resulting diseases are gene/mutation-specific although phenotypic overlap exists, and can be modified by sequence changes in interacting proteins. There have been significant advances in our understanding of the PR sensory cilium, and how mutations cause defective ciliogenesis or disease. In regards to therapy, however, advances have been slower and more variable. For the LCA-ciliopathies, early PR degeneration limits the window for corrective therapeutic intervention(s), resulting in modest and transient outcomes as therapy is initiated after the onset of degeneration. In contrast, there has been dramatic success in LCA-ciliopathy models [mouse and dogs with RPGR-X linked RP (XLRP) and RPGRIP1]. This highlights the complexity of the disorders, and the need to better understand the therapeutic options and barriers to optimizing treatment outcomes.

Disease-relevant animal models have proven crucial in developing and validating new retinal therapies. For LCA-ciliopathies there are several naturally occurring or genetically engineered mice, but only 3 large animal models—CEP290 cat, and NPHP4 and NPHP5 dogs. The CEP290 cat model bears a hypomorphic allele, and thus resembles late-onset RP rather than LCA; the NPHP4 dog is an LCA-model that exists only in the pet population, and is not available for research. A canine NPHP5 ciliopathy model from the University of Pennsylvania is particularly useful as it recapitulates the disease in patients with 5 major cilopathies—CEP290, RPGRIP1, Lebercilin, NPHP5, TULP1—in showing profound congenital retinal malfunction, preferential preservation of central cones, and a disease time course like that in man. The foveo-macular area of preservation in man is comparable to the visual streak that includes the fovea-like region in dogs; this region is slower to degenerate in NPHP5 dogs. This clearly identifiable region permits focal direct treatments via a subretinal route, or by intravitreal delivery once this route is optimized for clinical applications.

As well, the dog eye size is nearly comparable to the human so that issues of vector dosing can be assessed more accurately than in smaller animal species. By detailed characterization of the disease using in vivo imaging, functional, morphological and immunohistochemistry (IHC) methods, concrete disease metrics that reduce the interval between intervention and assessment are beginning to be established, thus expediting the time to translation of the basic research findings; e.g. successful initial outcome of treatment can be established within 7 wks. Finally, studies in the NPHP5 dog model are relevant for additional LCA-ciliopathies that feature selective central cone preservation, and the therapeutic questions addressed will be more broadly applicable.

No successful treatment for NPHP5-LCA is currently available to human patients suffering from this disease. What is needed is a treatment for NPHP5-LCA that is effective, safe and has long-term stability.

### SUMMARY OF THE INVENTION

In one aspect, a recombinant adeno-associated virus (AAV) is provided. The rAAV includes an AAV capsid protein and a nucleic acid sequence encoding a normal NPHP5 protein, or fragment thereof, under the control of regulatory sequences which express the NPHP5 in the photoreceptor cells of a subject. In one embodiment, the rAAV comprises an AAV8 capsid, or variant thereof. In another embodiment, the AAV8 capsid variant comprises a tyrosine to phenylalanine mutation. In another embodiment, the rAAV comprises an AAV5 capsid, or variant thereof. In yet another embodiment, the rAAV is a self-complementary AAV. In one embodiment, the regulatory sequences comprise a human GRK1 promoter. In another embodiment, the regulatory sequences comprise an IRBP promoter.

In another aspect, a method of preventing, arresting progression of or ameliorating vision loss associated with LCA-ciliopathy in a subject is provided. The method includes administering to the subject an effective concentration of a composition comprising a recombinant adeno-associated virus (AAV) carrying a nucleic acid sequence encoding a normal NPHP5 protein, or fragment thereof, under the control of regulatory sequences which express the NPHP5 in the photoreceptor cells of said subject, and a pharmaceutically acceptable carrier. In one embodiment, the method utilizes any of the compositions described herein.

In another embodiment, a method of treating or preventing LCA-ciliopathy in a subject in need thereof is provided. The method includes (a) identifying a subject having, or at risk of developing, LCA-ciliopathy; (b) performing genotypic analysis and identifying a mutation in the NPHP5 gene; (c) performing non-invasive retinal imaging and functional studies and identifying areas of retained photoreceptors that could be targeted for therapy; (d) administering to said subject an effective concentration of a composition comprising a recombinant virus carrying a nucleic acid sequence encoding a normal photoreceptor cell-specific gene under the control of a promoter sequence which expresses the product of said gene in said photoreceptor cells, and a pharmaceutically acceptable carrier, wherein said LCA-ciliopathy is prevented, arrested or ameliorated.

### BRIEF DESCRIPTION OF THE FIGURES

FIG. 1 is a series of electroretinographic traces (ERGs) demonstrating that treatment with AAV2/5-hIRBP-cNPHP5 or scAAV2/8-hGRK1-cNPHP5 rescues function of rods for at least 2.2 years. ERGs shown in the left column are those from an NPHP5 mutant dog treated with 1.5×10¹² vg/ml of AAV2/5-hIRBP-cNPHP5 at 5.7 weeks of age, as described in Example 1. ERGs shown in the center column are those from an NPHP5 mutant dog treated with 1.5×10¹¹ vg/ml of self-complementary (sc)AAV2/8 (Y733F)-GRK1-cNPHP5. ERGs shown in the right column are those from an NPHP5 mutant dog treated with 1.5×10¹² vg/ml of self-complementary (sc)AAV2/8 (Y733F)-GRK1-cNPHP5. From top to bottom, data is shown for the following ages: 13, 20, 32, 49, 65, 79, 99, and 125 weeks.

FIG. 2 is a series of electroretinographic traces (ERGs) demonstrating that treatment with AAV2/5-hIRBP-cNPHP5 or scAAV2/8-GRK1-cNPHP5 rescues function of cones for at least 2.2 years. ERGs shown in the left column are those from an NPHP5 mutant dog treated with 1.5×10¹² vg/ml of AAV2/5-IRBP-cNPHP5 at 5.7 weeks of age. ERGs shown in the center column are those from an NPHP5 mutant dog treated with 1.5×10¹¹ vg/ml of self-complementary (sc)AAV2/8 (Y733F)-GRK1-cNPHP5. ERGs shown in the right column are those from an NPHP5 mutant dog treated with 1.5×10¹² vg/ml of self-complementary (sc)AAV2/8 (Y733F)-GRK1-cNPHP5. From top to bottom, data is shown for the following ages: 13, 20, 32, 49, 65, 79, 99, and 125 weeks.

FIG. 3 shows (Left) a fundus photograph of a NPHP5 untreated dog retina, at 123 weeks of age. Diffuse hyperreflectivity and severe thinning of the retinal vasculature is shown indicating advanced retinal degeneration. Hyporeflectivity along the visual streak (which includes the area centralis) suggests less severe retinal degeneration in this region. Also shown (Right) is a composite infrared image of the same retina captured by confocal scanning laser ophthalmoscopy (cSLO) showing severe thinning of the retinal vasculature.

FIG. 4 are 30°×30° cSLO images (left) showing the location (arrow) of an optical coherence tomography (OCT) B scan in the temporal retina of an NPHP5 untreated dog eye, at 14, 33, 51 weeks of age. These images show progressive thinning of the outer nuclear layer (ONL) which contains the photoreceptor cells.

FIG. 5 are 30°×30° cSLO images (left) showing the location (arrow) of an optical coherence tomography (OCT) B scan in the temporal retina of an NPHP5 untreated dog eye, at 66, 79, 125 weeks of age. These images show progressive thinning of the outer nuclear layer (ONL) which contains the photoreceptor cells.

FIG. 6 are a fundus photograph (Left), infrared (center) and autofluorescence (right) mode composite images captured by confocal scanning laser ophthalmoscopy (cSLO) of an NPHP5 dog retina treated at 5.7 weeks of age with 1.5×10¹² vg/mL of AAV2/5-IRBP-cNPHP5, as described herein. Images taken at 124-125 weeks of age show preservation of retinal vasculature in the treated area while diffuse hyperreflectivity and severe thinning of the retinal vasculature indicative of advanced retinal degeneration is seen in the untreated areas.

FIG. 7 are 30°×30° cSLO images (left) showing the location (arrow) of an optical coherence tomography (OCT) B scan in the temporal retina of an NPHP5 dog retina treated at 5.7 weeks of age with 1.5×10¹² vg/mL of AAV2/5-IRBP-cNPHP5, as described herein. These images show preservation of the outer nuclear layer (ONL) which contains the photoreceptor cells at 14, 33, and 51 weeks of age.

FIG. 8 are 30°×30° cSLO images (left) showing the location (arrow) of an optical coherence tomography (OCT) B scan in the temporal retina of an NPHP5 dog retina treated at 5.7 weeks of age with 1.5×10¹² vg/mL of AAV2/5-IRBP-cNPHP5, as described herein. These images show preservation of the outer nuclear layer (ONL) which contains the photoreceptor cells at 66, 79, and 125 weeks of age.

FIG. 9 shows (Left) a fundus photograph of a NPHP5 untreated dog retina, at 123 weeks of age. Diffuse hyperreflectivity and severe thinning of the retinal vasculature is shown indicating advanced retinal degeneration. Hyporeflectivity along the visual streak (which includes the area centralis) suggests less severe retinal degeneration in this region. Also shown (Right) is an infrared composite image of the same retina obtained by confocal scanning laser ophthalmoscopy (cSLO) showing severe thinning of the retinal vasculature.

FIG. 10 are 30°×30° cSLO images (left) showing the location (arrow) of an optical coherence tomography (OCT) B scan in the temporal retina of an NPHP5 untreated dog eye, at 14, 33, 66 weeks of age. These images show progressive thinning of the outer nuclear layer (ONL) which contains the photoreceptor cells.

FIG. 11 are 30°×30° cSLO images (left) showing the location (arrow) of an optical coherence tomography (OCT) B scan in the temporal retina of an NPHP5 untreated dog eye, at 79, 97, and 125 weeks of age. These images show progressive thinning of the outer nuclear layer (ONL) which contains the photoreceptor cells.

FIG. 12 are a fundus photograph (Upper Left), infrared (Lower Left), and autofluorescence (right) mode composite images captured by confocal scanning laser ophthalmoscopy (cSLO) of an NPHP5 dog retina treated at 5.7 weeks of age with 1.5×10¹¹ vg/mL of scAAV2/8(Y733F)-GRK1-cNPHP5, as described herein. Images taken at 123-125 weeks of age show preservation of retinal vasculature in the treated area while diffuse hyperreflectivity and severe thinning of the retinal vasculature indicative of advanced retinal degeneration is seen in the untreated areas.

FIG. 13 are 30°×30° cSLO images (left) showing the location (arrow) of an optical coherence tomography (OCT) B scan in the temporal retina of an NPHP5 dog retina treated at 5.7 weeks of age with 1.5×10¹¹ vg/mL of scAAV2/8(Y733F)-GRK1-cNPHP5, as described herein. These images show preservation of the outer nuclear layer (ONL) which contains the photoreceptor cells at 14, 33, and 66 weeks of age.

FIG. 14 are 30°×30° cSLO images (left) showing the location (arrow) of an optical coherence tomography (OCT) B scan in the temporal retina of an NPHP5 dog retina treated at 5.7 weeks of age with 1.5×10¹¹ vg/mL of scAAV2/8(Y733F)-GRK1-cNPHP5, as described herein. These images show preservation of the outer nuclear layer (ONL) which contains the photoreceptor cells at 79, 97, and 125 weeks of age.

FIG. 15 shows (Left) a fundus photograph of a NPHP5 untreated dog retina, at 123 weeks of age. Diffuse hyperreflectivity and severe thinning of the retinal vasculature is shown indicating advanced retinal degeneration. Hyporeflectivity along the visual streak (which includes the area centralis) suggests less severe retinal degeneration in this region. Also shown (Right) is an infrared composite image of the same retina captured by confocal scanning laser ophthalmoscopy (cSLO) showing severe thinning of the retinal vasculature.

FIG. 16 are 30°×30° cSLO images (left) showing the location (arrow) of an optical coherence tomography (OCT) B scan in the temporal retina of an NPHP5 untreated dog eye, at 14, 33, 51, and 66 weeks of age. These images show progressive thinning of the outer nuclear layer (ONL) which contains the photoreceptor cells.

FIG. 17 are 30°×30° cSLO images (left) showing the location (arrow) of an optical coherence tomography (OCT) B scan in the temporal retina of an NPHP5 untreated dog eye, at 79, 97, and 125 weeks of age. These images show progressive thinning of the outer nuclear layer (ONL) which contains the photoreceptor cells.

FIG. 18 are a fundus photograph (Upper Left), infrared (Lower Left) and autofluorescence (right) mode composite images captured by confocal scanning laser ophthalmoscopy (cSLO) of an NPHP5 dog retina treated at 5.7 weeks of age with 1.5×10¹² vg/mL of scAAV2/8(Y733F)-GRK1-cNPHP5, as described herein. Images taken at 123-125 weeks of age show preservation of retinal vasculature in the treated area while diffuse hyperreflectivity and severe thinning of the retinal vasculature indicative of advanced retinal degeneration is seen in the untreated areas.

FIG. 19 are 30°×30° cSLO images (left) showing the location (arrow) of an optical coherence tomography (OCT) B scan in the temporal retina of an NPHP5 dog retina treated at 5.7 weeks of age with 1.5×10¹² vg/mL of scAAV2/8(Y733F)-GRK1-cNPHP5, as described herein. These images show preservation of the outer nuclear layer (ONL) which contains the photoreceptor cells at 14, 33, 51, and 66 weeks of age.

FIG. 20 are 30°×30° cSLO images (left) showing the location (arrow) of an optical coherence tomography (OCT) B scan in the temporal retina of an NPHP5 dog retina treated at 5.7 weeks of age with 1.5×10¹² vg/mL of scAAV2/8(Y733F)-GRK1-cNPHP5, as described herein. These images show preservation of the outer nuclear layer (ONL) which contains the photoreceptor cells at 79, 97, and 125 weeks of age.

FIG. 21 are topographical maps of outer nuclear layer (ONL) thickness generated from post-acquisition processing of overlapping raster OCT B scans at 14, 33, 51, and 66 wks of age in three NPHP5 dog retinas treated at 5.7 wks of age. Top row shows progression of ONL thickness in a retina treated with 1.5×10¹² vg/mL of AAV2/5-IRBP-cNPHP5, as described herein. Middle row shows progression of ONL thickness in a retina treated at 5.7 weeks of age with 1.5×10¹¹ vg/mL of scAAV2/8(Y733F)-GRK1-cNPHP5, as described herein. Lower row shows progression of ONL thickness in a retina treated at 5.7 weeks of age with 1.5×10¹² vg/mL of scAAV2/8(Y733F)-GRK1-cNPHP5, as described herein. A positive rescue effect was seen in the treated area (demarcated by a dark contour line) in all three dogs. A better ONL rescue effect was seen in the animal treated with 1.5×10¹² vg/mL of scAAV2/8(Y733F)-GRK1-cNPHP5.

FIG. 22 is a series of electroretinographic traces (ERGs) demonstrating in NPHP5 mutant dogs at 13 weeks of age response to treatment with three different vector constructs delivered at 5.7 weeks. Treatment with 4.74×10¹² vg/ml of self-complementary (sc)AAV2/8 (Y733F)-GRK1-cNPHP5 led to prominent rod, mixed rod-cone, and cone ERG rescue (Left column). Treatment with 1.5 or 4.74×10¹² vg/ml of scAAV2/8 (Y733F)-GRK1-hNPHP5 led to mild cone ERG rescue (Central column). Treatment with 1.5 or 4.74×10¹² vg/ml of scAAV2/8mut C&G+T494V-GRK1-cNPHP5 led to prominent rod, mixed rod-cone, and cone ERG rescue (Right column).

FIG. 23 is a series of electroretinographic traces (ERGs) demonstrating that treatment with 4.74×10¹² vg/ml of scAAV2/8mut C&G+T494V-GRK1-cNPHP5 rescues rod function in two NPHP5 mutant dogs injected after the onset of retinal degeneration at 8.6 weeks of age From top to bottom, data is shown for the following ages: approx. 33, 52, and 67 weeks.

FIG. 24 is a series of electroretinographic traces (ERGs) demonstrating that treatment with 4.74×10¹² vg/ml of scAAV2/8mut C&G+T494V-GRK1-cNPHP5 rescues cone function in two NPHP5 mutant dogs injected after the onset of retinal degeneration at 8.6 weeks of age From top to bottom, data is shown for the following ages: approx. 33, 52, and 67 weeks.

FIG. 25 shows (Top Left) a fundus photograph of a NPHP5 untreated dog retina, at 65 weeks of age. Diffuse hyperreflectivity and severe thinning of the retinal vasculature is shown indicating advanced retinal degeneration. Also shown are an infrared (Bottom Left) and autofluorescence (Right) composite images of the same retina acquired by confocal scanning laser ophthalmoscopy (cSLO) showing severe thinning of the retinal vasculature.

FIG. 26 are 30°×30° cSLO images (left) showing the location (arrow) of an optical coherence tomography (OCT) B scan in the temporal retina of an NPHP5 untreated dog eye at 7, 20, 49 and 65 weeks of age. These images show progressive thinning of the outer nuclear layer (ONL) which contains the photoreceptor cells.

FIG. 27 are a fundus photograph immediately after injection (Top Left), a fundus photograph at 60 wks of age (Top Center), and infrared (bottom Left) and autofluorescence (right) mode composite images captured by confocal scanning laser ophthalmoscopy (cSLO) of an NPHP5 dog retina treated at 8.6 weeks of age with 4.74×10¹² vg/mL of scAAV2/8 mut C&G+T494V-GRK1-cNPHP5, as described herein. Images taken at 60-65 weeks of age show preservation of retinal vasculature in the treated area while diffuse hyperreflectivity and severe thinning of the retinal vasculature indicative of advanced retinal degeneration is seen in the surrounding untreated areas.

FIG. 28 are 30°×30° cSLO images (left) showing the location (arrow) of an optical coherence tomography (OCT) B scan in the temporal retina of an NPHP5 dog retina treated at 8.6 weeks of age with 4.74×10¹² vg/mL of scAAV2/8 mut C&G+T494V-GRK1-cNPHP5, as described herein. These images show the outer nuclear layer (ONL) which contains the photoreceptor cells at 7 weeks of age (before treatment) and its preservation after treatment at 20, 49, and 65 weeks of age.

FIG. 29 is a series of electroretinographic traces (ERGs) demonstrating that treatment with 4.74×10¹² vg/ml of scAAV2/8mut C&G+T494V-GRK1-cNPHP5 recovers rod function in an NPHP5 mutant dog injected at a later stage of retinal degeneration (13.9 weeks of age). From top to bottom, data is shown for the following ages: approx. 13.9 (pre-injection), and at 20, 28, and 51 weeks age (post-injection).

FIG. 30 is a series of electroretinographic traces (ERGs) demonstrating that treatment with 4.74×10¹² vg/ml of scAAV2/8mut C&G+T494V-GRK1-cNPHP5 recovers cone function in an NPHP5 mutant dog injected at a later stage of retinal degeneration (13.9 weeks of age). From top to bottom, data is shown for the following ages: approx. 13.9 (pre-injection), and at 20, 21, and 51 weeks age (post-injection).

FIG. 31 shows (Top Left) a fundus photograph of an NPHP5 untreated dog retina, at 50 weeks of age. Diffuse hyperreflectivity and severe thinning of the retinal vasculature is shown indicating advanced retinal degeneration. Also shown are an infrared (Bottom Left) and autofluorescence composite images (Right) of the same retina captured by confocal scanning laser ophthalmoscopy (cSLO) showing at 53 weeks of age severe thinning of the retinal vasculature.

FIG. 32 are 30°×30° cSLO images (left) showing the location (arrow) of an optical coherence tomography (OCT) B scan in the temporal retina of an NPHP5 untreated dog eye at 13, 30, and 53 weeks of age. These images show progressive thinning of the outer nuclear layer (ONL) which contains the photoreceptor cells.

FIG. 33 are a fundus photograph immediately after injection (Top Left), a fundus photograph at 50 wks of age (Top Right), and infrared (bottom Left) and autofluorescence (bottom right) mode composite images captured by confocal scanning laser ophthalmoscopy (cSLO) of an NPHP5 dog retina treated at 13.9 weeks of age with 4.74×10¹² vg/mL of scAAV2/8 mut C&G+T494V-GRK1-cNPHP5, as described herein. Images taken at 50-53 weeks of age show preservation of retinal vasculature in the treated area and retention of a normal-appearing tapetal reflectivity.

FIG. 34 are 30°×30° cSLO images (left) showing the location (arrow) of an optical coherence tomography (OCT) B scan in the temporal retina of an NPHP5 dog retina treated at 13.9 weeks of age with 4.74×10¹² vg/mL of scAAV2/8 mut C&G+T494V-GRK1-cNPHP5, as described herein. These images show the outer nuclear layer (ONL) which contains the photoreceptor cells at 13 weeks of age (before treatment) and its preservation after treatment at 30, and 53 weeks of age.

### DETAILED DESCRIPTION OF THE INVENTION

The present invention relates to various compositions and treatment methods utilizing the same comprising an effective concentration of a recombinant adeno-associated virus (rAAV) carrying a nucleic acid sequence encoding a normal NPHP5 protein, or fragment thereof, under the control of regulatory sequences which direct expression of the protein in the subject's ocular cells, formulated with a carrier and additional components suitable for injection. The treatment methods are directed to ocular disorders and associated conditions related thereto.

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

The terms “a” or “an” refers to one or more, for example, “a gene” is understood to represent one or more such genes. As such, the terms “a” (or “an”), “one or more,” and “at least one” are used interchangeably herein. As used herein, the term “about” means a variability of 10% from the reference given, unless otherwise specified.

With regard to the following description, it is intended that each of the compositions herein described, is useful, in another embodiment, in the methods of the invention. In addition, it is also intended that each of the compositions herein described as useful in the methods, is, in another embodiment, itself an embodiment of the invention. While various embodiments in the specification are presented using “comprising” language, under other circumstances, a related embodiment is also intended to be interpreted and described using “consisting of” or “consisting essentially of” language.

### A. LCA-CILIOPATHY

The ciliopathies form a class of genetic disease which result in either abnormal formation or function of cilia. As cilia are a component of almost all vertebrate cells, cilia dysfunction can manifest as a constellation of features that include characteristically, retinal degeneration, renal disease and cerebral anomalies. Senior-Loken syndrome is an autosomal recessive oculo-renal condition. The 2 major features of Senior-Loken syndrome are the cystic kidney disease known as nephronophthisis (NPHP) and an early childhood-onset retinal degeneration known as Leber congenital amaurosis (LCA). To date, Senior-Loken syndrome has been associated with mutations in 5 of the 10 NPHP genes. NPHP6 is thought to form a functional complex with NPHP5 (OMIM 609237) and knockdown of either of these genes in zebrafish embryos leads to a syndromic disease with ocular and systemic manifestations. Certain mutations in NPHP5 have been shown to cause LCA (Stone et al, Variations in NPHP5 in Patients With Nonsyndromic Leber Congenital Amaurosis and Senior-Loken Syndrome, Arch Ophthalmol. 2011 January; 129(1): 81-87, which is incorporated herein by reference), in the absence of Senior Loken syndrome.

As used herein, the term “LCA-ciliopathy” refers to any condition which shows retinal degeneration similar to that shown in LCA. For example, LCA is typically characterized by nystagmus, sluggish or absent pupillary responses, and severe vision loss or blindness. In one embodiment, LCA-ciliopathy refers to a subset of one of the recognized 18 types of LCA (OMIM.com). In another embodiment, LCA-ciliopathy refers to retinal disease associated with Senior-Loken syndrome. In another embodiment, LCA-ciliopathy refers to retinal disease associated with Bardet-Biedl syndrome, Meckel-Gruber syndrome, Joubert syndrome, or nephronophthisis. In one embodiment, LCA-ciliopathy refers to LCA associated with NPHP5 mutation. In another embodiment, LCA-ciliopathy refers to retinitis pigmentosa associated with NPHP5 mutation. In yet another embodiment, LCA-ciliopathy refers to non-syndromic LCA.

### B. THE MAMMALIAN SUBJECT

As used herein, the term “mammalian subject” or “subject” includes any mammal in need of these methods of treatment or prophylaxis, including particularly humans. Other mammals in need of such treatment or prophylaxis include dogs, cats, or other domesticated animals, horses, livestock, laboratory animals, including non-human primates, etc. The subject may be male or female. In one embodiment, the subject has, or is at risk of developing, Leber congenital amaurosis (LCA) or a LCA-ciliopathy. In another embodiment, the subject has or is at risk of developing a LCA-ciliopathy associated with a mutation in NPHP5. In one embodiment, the subject has or is at risk of developing Senior-Loken syndrome.

In another embodiment, the subject has shown clinical signs of LCA-ciliopathy. Clinical signs of LCA-ciliopathy include, but are not limited to, nystagmus, decreased peripheral vision, decreased central (reading) vision, decreased night vision, loss of color perception, reduction in visual acuity, decreased photoreceptor function, pigmentary changes. In another embodiment, the subject has been diagnosed with LCA-ciliopathy. In yet another embodiment, the subject has not yet shown clinical signs of LCA-ciliopathy.

In yet another embodiment, the subject has 10% or more photoreceptor damage/loss. In another embodiment, the subject has 20% or more photoreceptor damage/loss. In another embodiment, the subject has 30% or more photoreceptor damage/loss. In another embodiment, the subject has 40% or more photoreceptor damage/loss. In another embodiment, the subject has 50% or more photoreceptor damage/loss. In another embodiment, the subject has 60% or more photoreceptor damage/loss. In another embodiment, the subject has 70% or more photoreceptor damage/loss. In another embodiment, the subject has 80% or more photoreceptor damage/loss. In another embodiment, the subject has 90% or more photoreceptor damage/loss.

In one another embodiment, the subject has 10% or more rod and/or cone function damage/loss. In one another embodiment, the subject has 20% or more rod and/or cone function damage/loss. In one another embodiment, the subject has 30% or more rod and/or cone function damage/loss. In one another embodiment, the subject has 40% or more rod and/or cone function damage/loss. In one another embodiment, the subject has 50% or more rod and/or cone function damage/loss. In one another embodiment, the subject has 60% or more rod and/or cone function damage/loss. In one another embodiment, the subject has 70% or more rod and/or cone function damage/loss. In one another embodiment, the subject has 80% or more rod and/or cone function damage/loss. In one another embodiment, the subject has 90% or more rod and/or cone function damage/loss.

### C. NPHP5

Nephrocystin 5 (NPHP5) is a 598 amino acid protein having a molecular mass of 69 kD. Also called IQ Motif-Containing protein B1 (IQCB1), NPHP5 is highly conserved in higher eukaryotes and possesses a putative coiled-coil and IQ calmodulin (CaM)-binding motifs of unknown function. See, Barbelanne et al, Hum Mol Genet. 2013 Jun. 15; 22(12): 2482-2494, which is incorporated herein by reference.

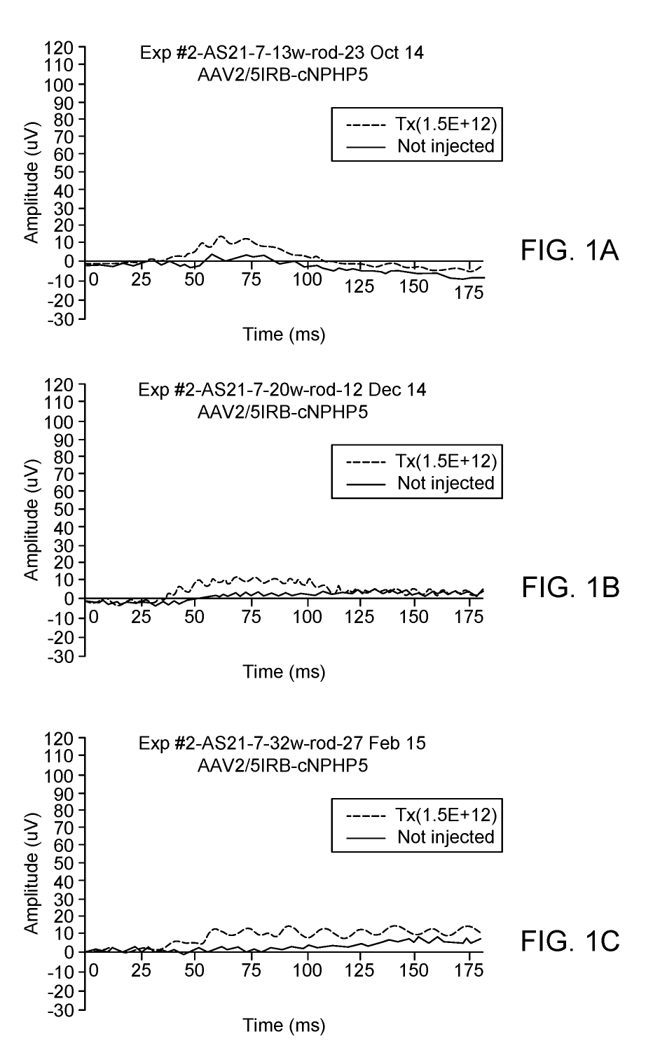

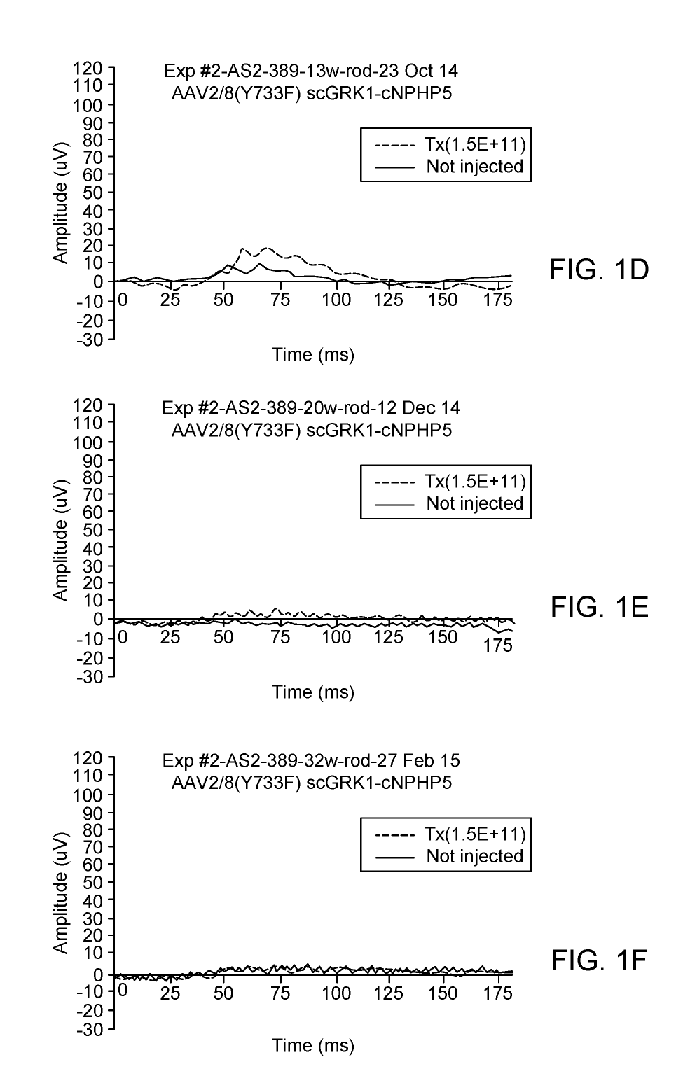

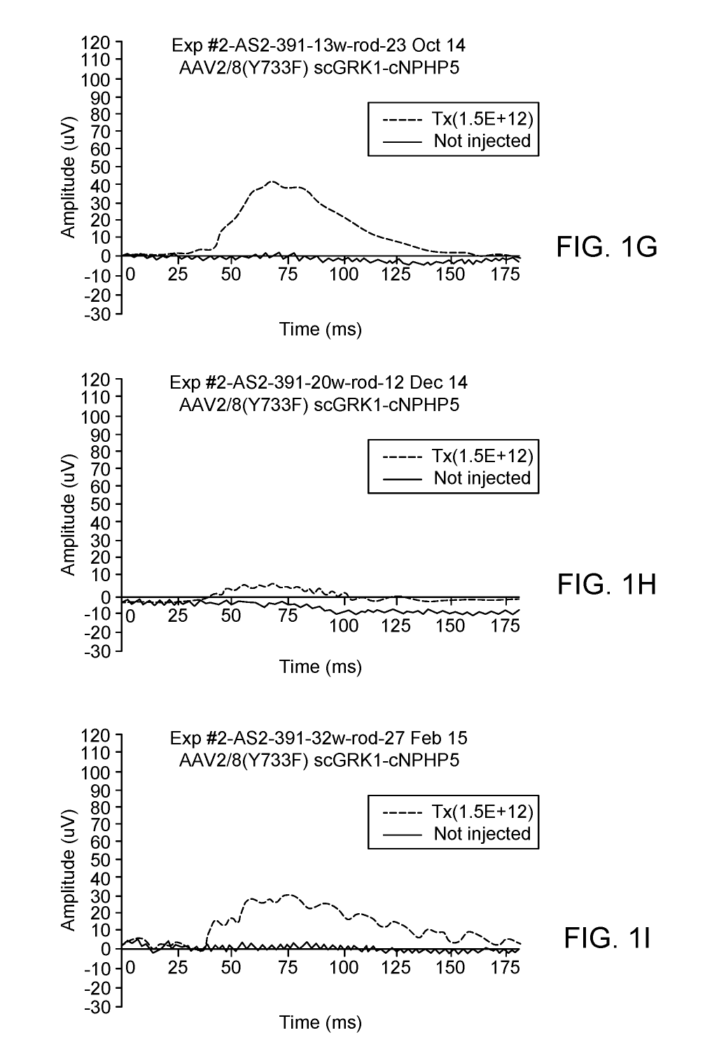

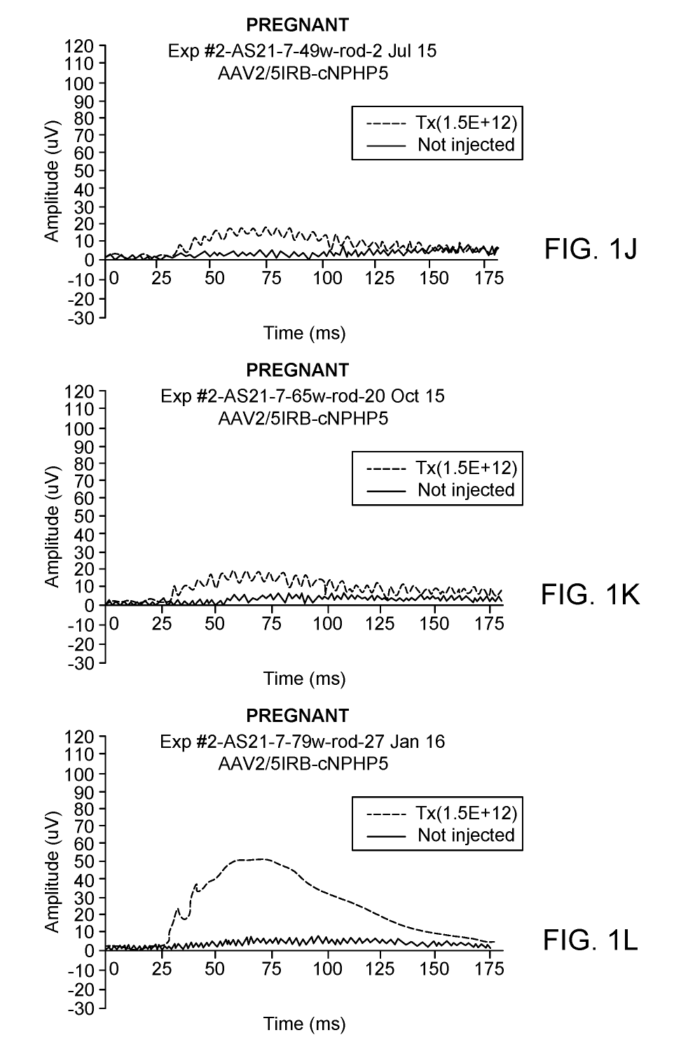

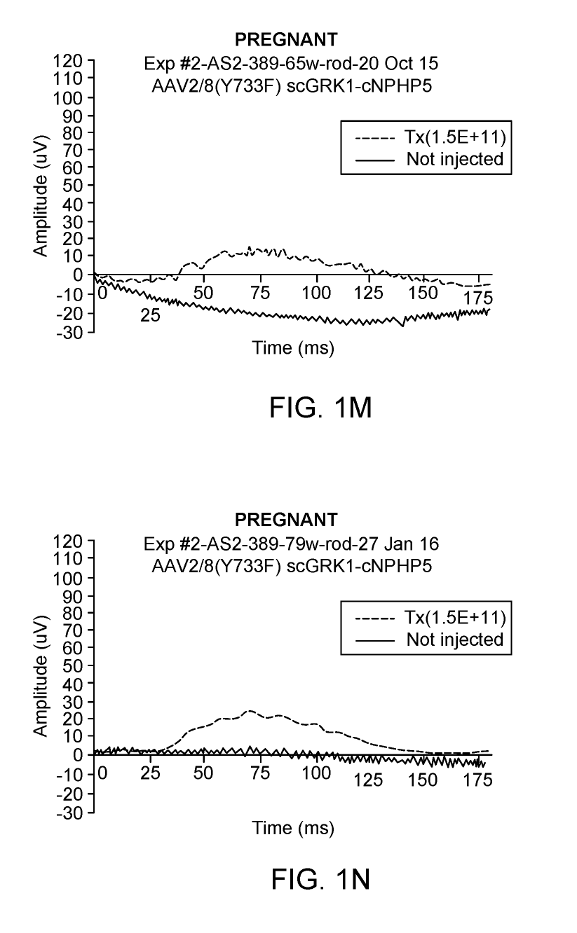

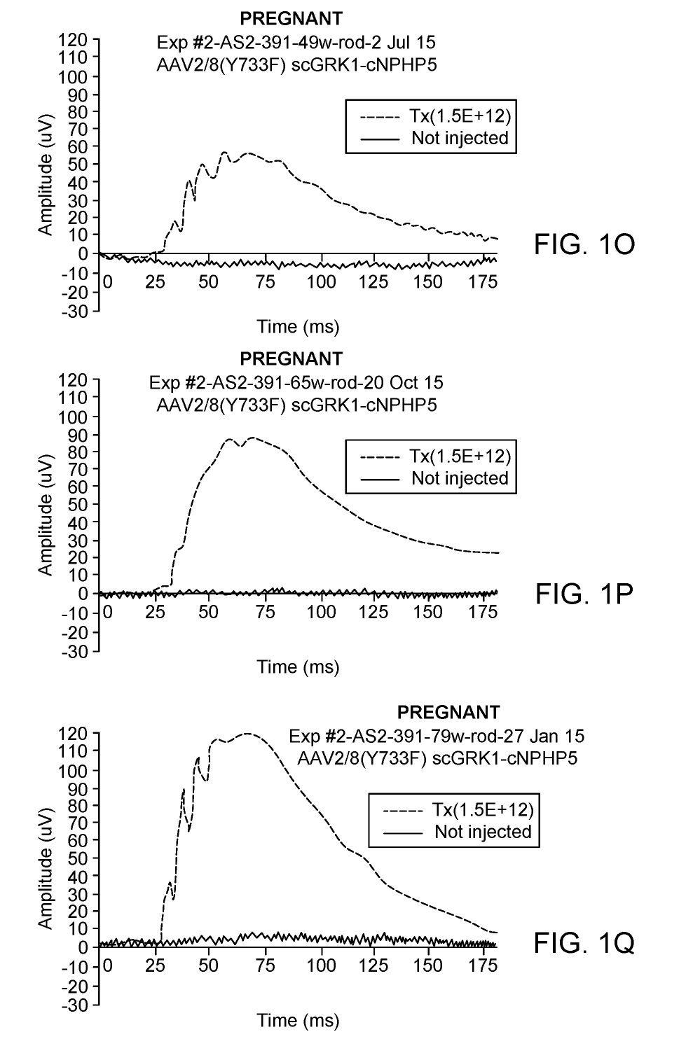

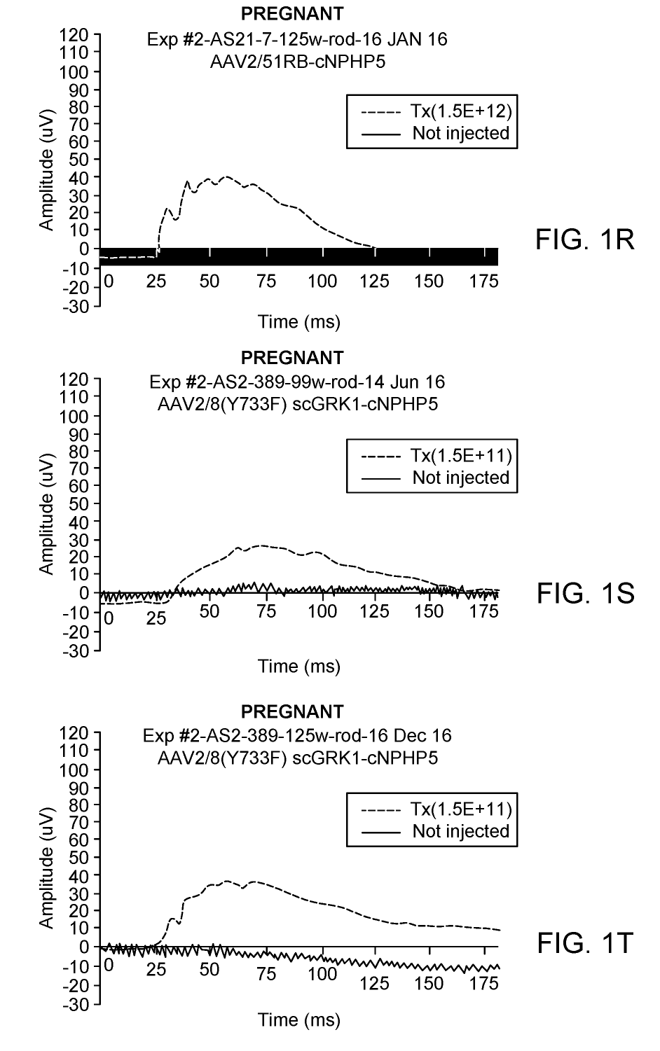

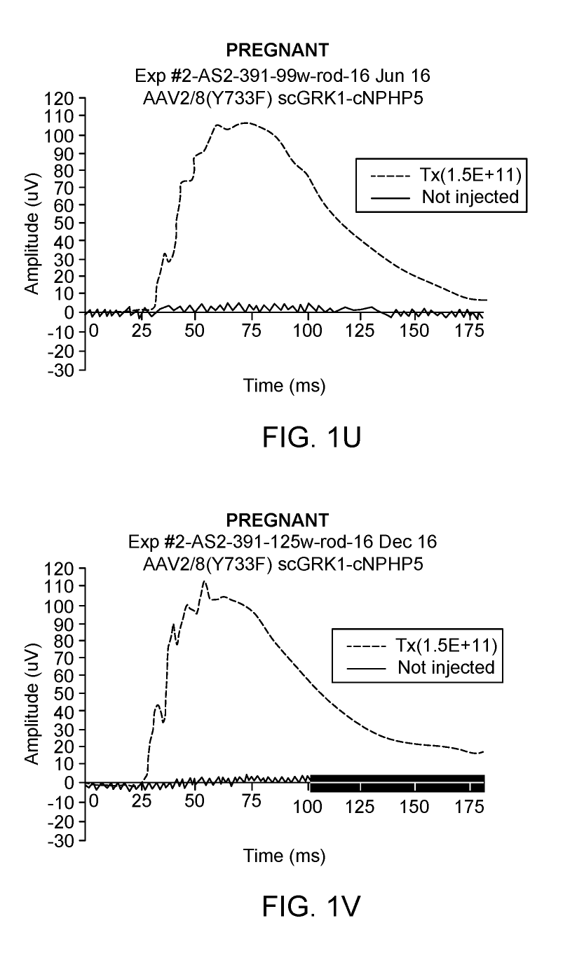

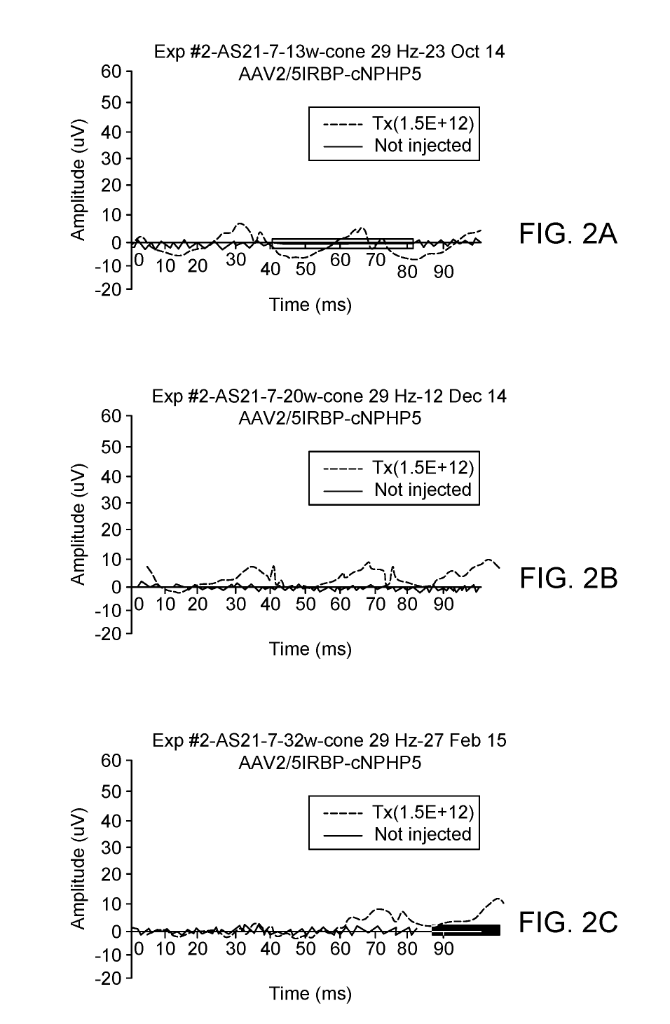

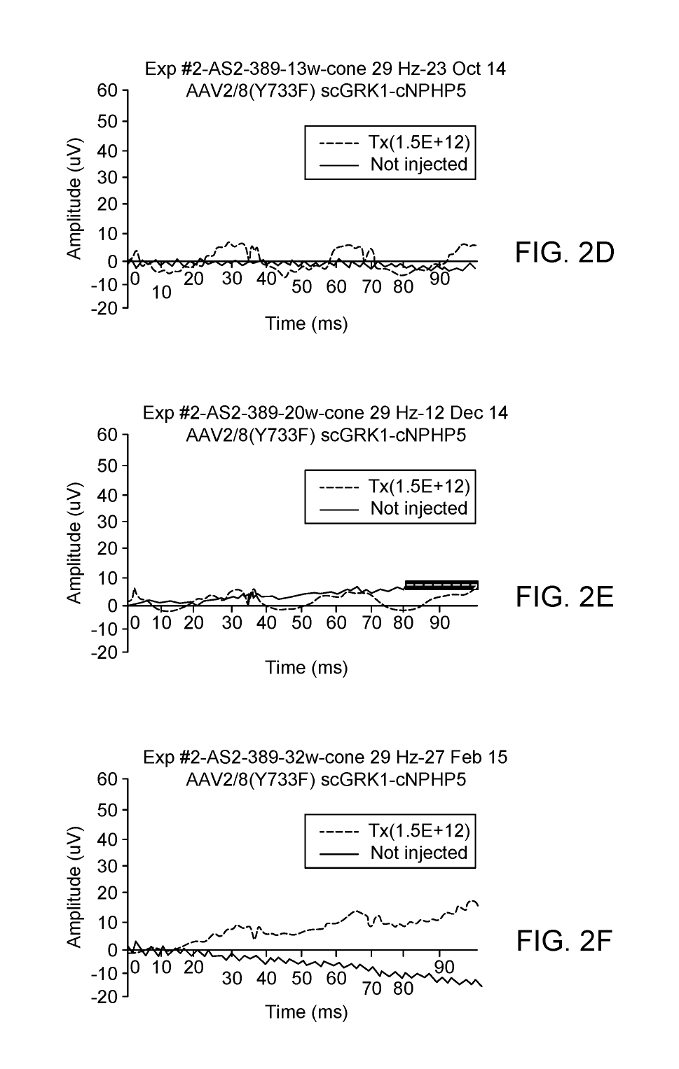

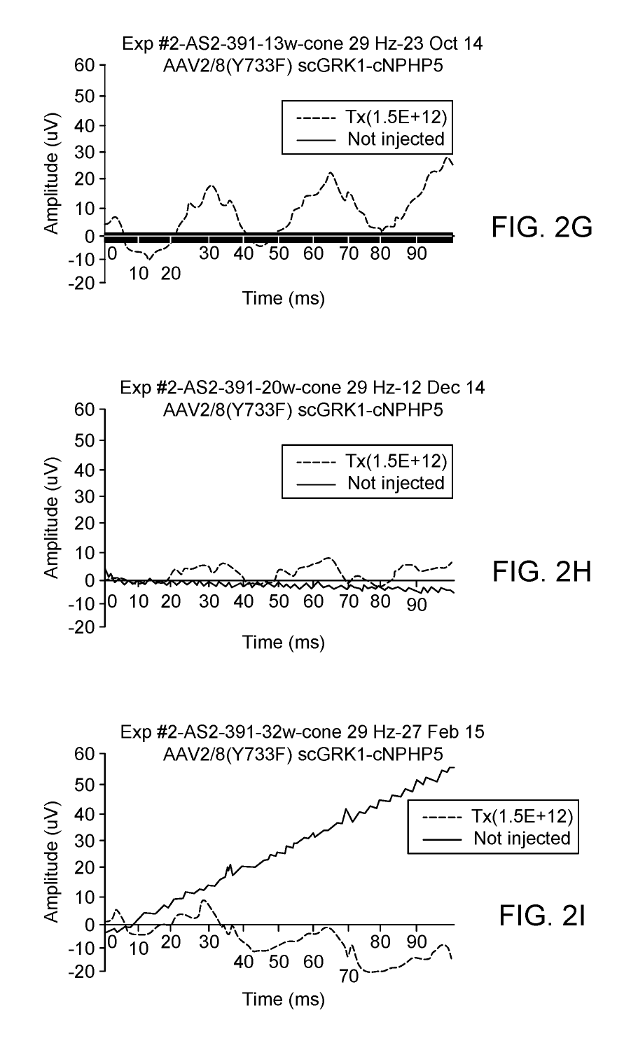

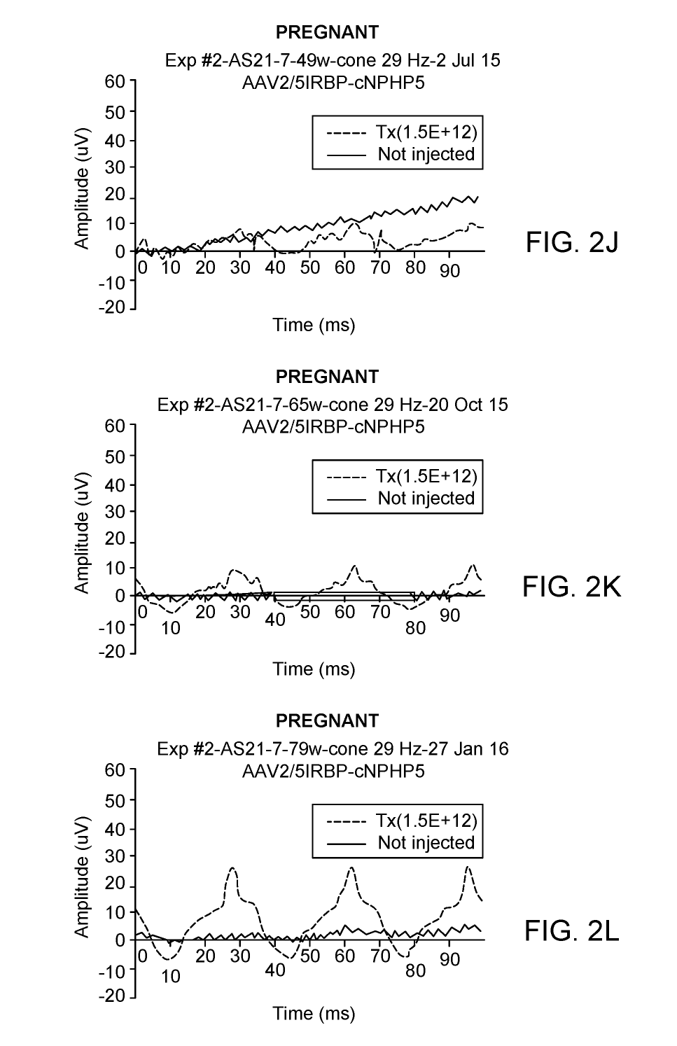

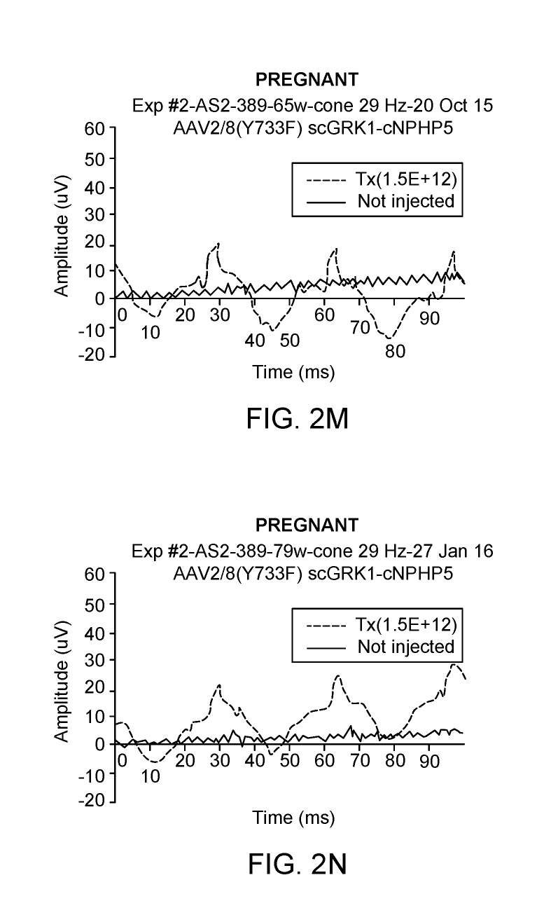

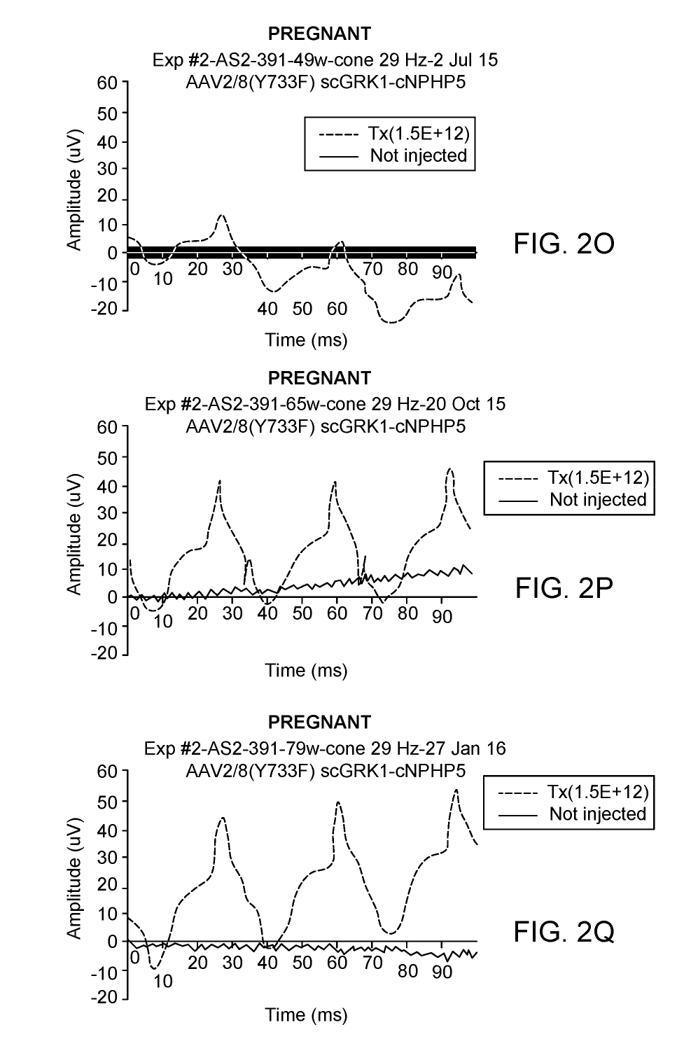

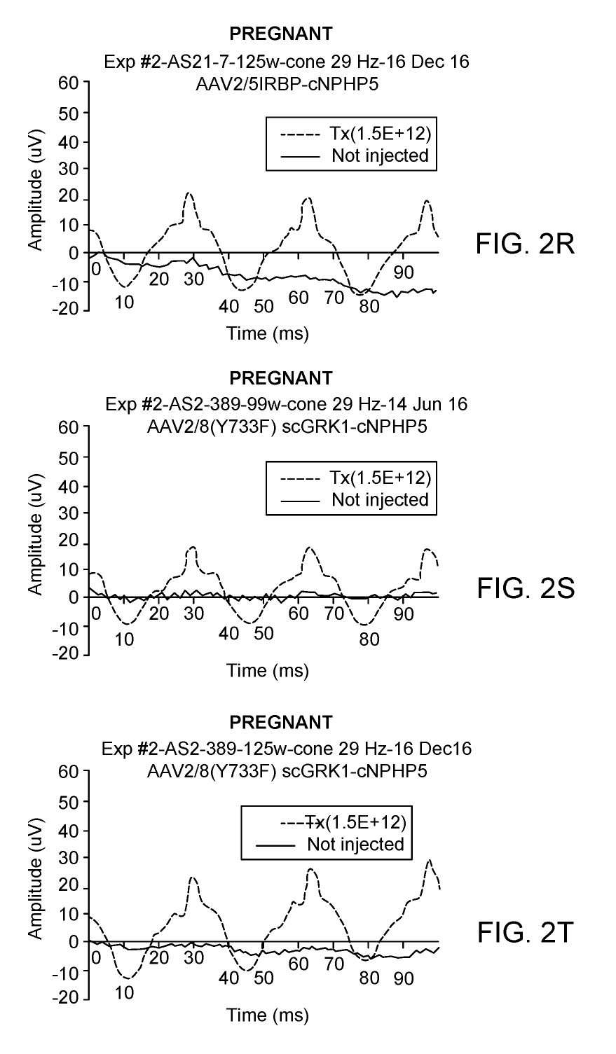

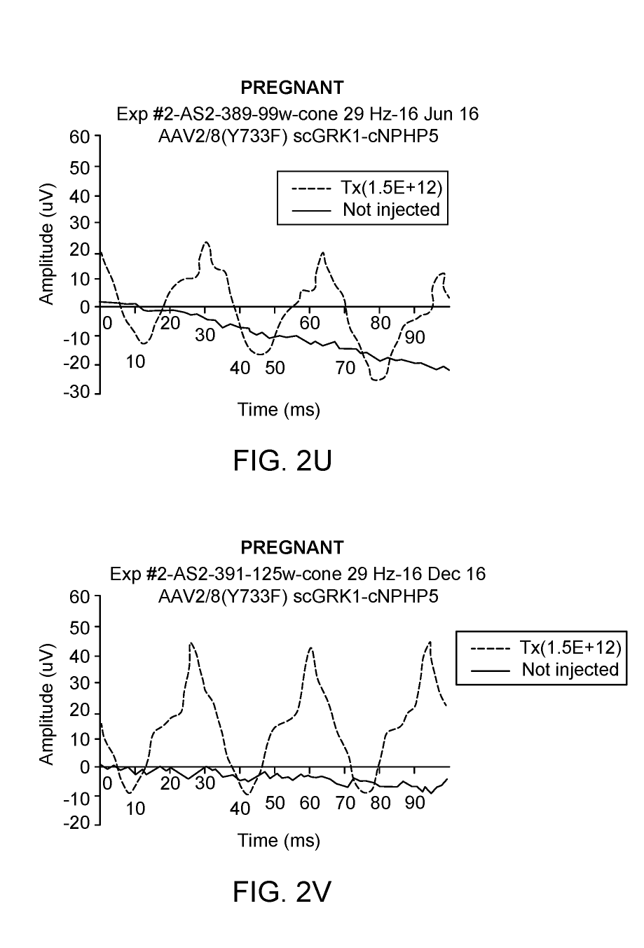

The NPHP5 protein shows 89% human-dog identity. A NPHP5 Leber congenital amaurosis canine model is available (ARVO 2015 Annual Meeting Abstracts, Aguirre et al, Photoreceptor development, degeneration and retinal gene expression in the canine NPHP5 Leber congenital amaurosis model, which is incorporated herein by reference; and Goldstein O, Mezey J G, Schweitzer P A, Boyko A R, Gao C, Bustamante C D, Jordan J A, Aguirre G D, Acland G M. 2013. IQCB1 and PDE6B mutations cause similar early onset retinal degenerations in two closely related terrier dog breeds. Invest Ophthalmol Vis Sci; 54:7005-7019, which is incorporated herein by reference.) and is utilized in the Examples described herein. In NPHP5 dogs, the mutation is a cytosine insertion in exon 10, a frame shift between aa 318-330, and truncation of the terminal 268 aa that eliminates the second of two BBS binding domains, and the CEP290 binding domain. C-terminal truncation mutations generally apparent in NPHP5-LCA patients. In dogs the disease is a nonsyndromic LCA as brain and kidney structures and renal function are normal (up to 9.5 yrs of age). Nonsyndromic LCA also occurs in patients, although more commonly it is expressed as a retinal/renal disease (Senior-Loken syndrome). The mutant retina in NPHP5 dogs develops abnormally, and degeneration, based on TUNEL labeling, peaks at 6 wks, and then declines to a constant but lower rate. Despite the relative structural rod preservation, rod responses are abnormal and markedly reduced in amplitude by 6 wks, and nearly absent by 14 wks (FIG. 1; not injected). Cone responses are not recordable at any time (FIG. 2; not injected). The dissociation between the rod structural and functional abnormalities is suggestive of defects in ciliary trafficking. The absence of cone-mediated responses correlates more directly with structural abnormalities, as the majority of cone OS are absent early, and most of the IS and remaining OS are lost by 14 wks (FIG. 2). What remains are cone cell bodies, nuclei, and distinct axons and pedicles. IHC analysis at 6 and 14 wks showed that PR sensory cilium markers, e.g. MAP9, acetylated tubulin, rootletin, clearly label this structure, an indication that these form. The rod and cone OS present have distinct labeling with opsin Abs (rod, blue, red/green), although mislocalization into IS and ONL also occurs. The protein sequence of native human NPHP5 is shown in SEQ ID NO: 1. The protein sequence of native canine NPHP5 is shown in SEQ ID NO: 2.

In one aspect the method employs a nucleic acid sequence encoding a normal NPHP5 protein, or fragment thereof. The term “NPHP5” as used herein, refers to the full length protein itself or a functional fragment, or variant thereof, as further defined below. The nucleic acid sequence encoding a normal NPHP5 protein may be derived from any mammal which natively expresses the NPHP5 protein, or homolog thereof. In another embodiment, the NPHP5 protein sequence is derived from the same mammal that the composition is intended to treat. In one embodiment, the NPHP5 is derived from a human. In another embodiment, the NPHP5 is derived from a canine.

In one embodiment, the NPHP5 protein sequence is that shown in SEQ ID NO: 1. In another embodiment, the NPHP5 protein sequence is that shown in SEQ ID NO: 2. In another embodiment, the NPHP5 protein sequence is a functional fragment of a native NPHP5 protein. By the term “fragment” or “functional fragment”, it is meant any fragment that retains the function of the full length protein, although not necessarily at the same level of expression or activity.

In another embodiment, the NPHP5 protein sequence is a variant which shares at least 80% identity with a native NPHP5 protein. In another embodiment, the NPHP5 protein sequence shares at least 85% identity with a native NPHP5 protein. In another embodiment, the NPHP5 protein sequence shares at least 90% identity with a native NPHP5 protein. In another embodiment, the NPHP5 protein sequence shares at least 91% identity with a native NPHP5 protein. In another embodiment, the NPHP5 protein sequence shares at least 92% identity with a native NPHP5 protein. In another embodiment, the NPHP5 protein sequence shares at least 93% identity with a native NPHP5 protein. In another embodiment, the NPHP5 protein sequence shares at least 94% identity with a native NPHP5 protein. In another embodiment, the NPHP5 protein sequence shares at least 95% identity with a native NPHP5 protein. In another embodiment, the NPHP5 protein sequence shares at least 96% identity with a native NPHP5 protein. In another embodiment, the NPHP5 protein sequence shares at least 97% identity with a native NPHP5 protein. In another embodiment, the NPHP5 protein sequence shares at least 98% identity with a native NPHP5 protein. In another embodiment, the NPHP5 protein sequence shares at least 99% identity with a native NPHP5 protein.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 70 amino acids to about 100 amino acids, or a peptide fragment thereof or the corresponding nucleic acid sequence coding sequencers. A suitable amino acid fragment may be at least about 8 amino acids in length, and may be up to about 150 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

In other embodiments, certain modifications are made to the NPHP5 coding sequence in order to enhance the expression in the target cell. Such modifications include codon optimization, (see, e.g., U.S. Pat. Nos. 7,561,972; 7,561,973; and 7,888,112, incorporated herein by reference) and conversion of the sequence surrounding the translational start site to a consensus Kozak sequence: gccRccATGR. See, Kozak et al, Nucleic Acids Res. 15 (20): 8125-8148, incorporated herein by reference.

In one embodiment, the coding sequences are designed for optimal expression using codon optimization. Codon-optimized coding regions can be designed by various different methods. This optimization may be performed using methods which are available on-line, published methods, or a company which provides codon optimizing services. One codon optimizing method is described, e.g., in International Patent Application Pub. No. WO 2015/012924, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan.

In addition, gene synthesis is readily available commercially. In one embodiment, the native NPHP5 coding sequence is the human coding sequence shown in SEQ ID NO: 3, or a variant thereof. In one embodiment, the native NPHP5 coding sequence is the canine coding sequence shown in SEQ ID NO: 4 (also known by accession number KF366421), or a variant thereof. In one embodiment, the NPHP5 coding sequence is a variant which shares at least 60% identity with a native NPHP5 coding sequence. In another embodiment, the NPHP5 coding sequence shares at least 65% identity with a native NPHP5 coding sequence. In another embodiment, the NPHP5 coding sequence shares at least 70% identity with a native NPHP5 coding sequence. In another embodiment, the NPHP5 coding sequence shares at least 75% identity with a native NPHP5 coding sequence. In another embodiment, the NPHP5 coding sequence shares at least 70, 71, 72, 73, 74, 75, 76, 77, 78, 79, 80, 81, 82, 83, 84, 85, 86, 87, 88, 89 or 90 or greater % identity with a native NPHP5 coding sequence.

The terms “percent (%) identity”, “sequence identity”, “percent sequence identity”, or “percent identical” in the context of nucleic acid sequences refers to the bases in the two sequences which are the same when aligned for correspondence. The length of sequence identity comparison may be over the full-length of the genome, the full-length of a gene coding sequence, or a fragment of at least about 100 to 150 nucleotides, or as desired. However, identity among smaller fragments, e.g. of at least about nine nucleotides, usually at least about 20 to 24 nucleotides, at least about 28 to 32 nucleotides, at least about 36 or more nucleotides, may also be desired. Multiple sequence alignment programs are also available for nucleic acid sequences. Examples of such programs include, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

### D. AAV VECTORS AND COMPOSITIONS

In certain embodiments of this invention, the NPHP5 nucleic acid sequence is delivered to the ocular cells in need of treatment by means of a viral vector, of which many are known and available in the art. For delivery to the ocular cells, the therapeutic vector is desirably non-toxic, non-immunogenic, easy to produce, and efficient in protecting and delivering DNA into the target cells. As used herein, the term “ocular cells” refers to any cell in, or associated with the function of, the eye. The term may refer to any one or more of photoreceptor cells, including rod, cone and photosensitive ganglion cells, retinal pigment epithelium (RPE) cells, Mueller cells, bipolar cells, horizontal cells, amacrine cells. In one embodiment, the ocular cells are the photoreceptor cells. In another embodiment, the ocular cells are the rod and cone cells. In yet another embodiment, the ocular cells are the cone cells.

A “vector” as used herein is a nucleic acid molecule into which an exogenous or heterologous or engineered nucleic acid transgene may be inserted which can then be introduced into an appropriate host cell. Vectors preferably have one or more origin of replication, and one or more site into which the recombinant DNA can be inserted.

Vectors often have convenient means by which cells with vectors can be selected from those without, e.g., they encode drug resistance genes. Common vectors include plasmids, viral genomes, and (primarily in yeast and bacteria) “artificial chromosomes.”

“Virus vectors” are defined as replication defective viruses containing the exogenous or heterologous NPHP5 nucleic acid transgene. In one embodiment, an expression cassette as described herein may be engineered onto a plasmid which is used for drug delivery or for production of a viral vector. Suitable viral vectors are preferably replication defective and selected from amongst those which target ocular cells. Viral vectors may include any virus suitable for gene therapy, including but not limited to adenovirus; herpes virus; lentivirus; retrovirus; parvovirus, etc. However, for ease of understanding, the adeno-associated virus is referenced herein as an exemplary virus vector.

In one particular embodiment, the viral vector is an adeno-associated virus vector. In another embodiment, the invention provides a therapeutic composition comprising an adeno-associated viral vector comprising an NPHP5 coding sequence operatively linked to expression control sequences. In one embodiment, the NPHP5 coding sequence is shown in SEQ ID NO: 3. In another embodiment, the NPHP5 coding sequence is shown in SEQ ID NO: 4. In another embodiment, the NPHP5 coding sequence is a codon optimized sequence of SEQ ID NO: 3. In another embodiment, the NPHP5 coding sequence is a codon optimized sequence of SEQ ID NO: 4.

As used herein, the term “operably linked” or “operatively associated” refers to both expression control sequences that are contiguous with the nucleic acid sequence encoding the NPHP5 and/or expression control sequences that act in trans or at a distance to control the transcription and expression thereof.

The term “AAV” or “AAV serotype” as used herein refers to the dozens of naturally occurring and available adeno-associated viruses, as well as artificial AAVs. An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. AAVs may be selected as sources for capsids of AAV viral vectors as identified above. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. No. 7,790,449 and U.S. Pat. No. 7,282,199 (AAV8), WO 2005/033321 and U.S. Pat. No. 7,906,111 (AAV9), and WO 2006/110689, and WO 2003/042397 (rh.10). These documents also describe other AAV which may be selected for generating AAV and are incorporated by reference.

In some embodiments, an AAV cap for use in the viral vector can be generated by mutagenesis (i.e., by insertions, deletions, or substitutions) of one of the aforementioned AAV capsids or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned Caps.

Among the AAVs isolated or engineered from human or non-human primates (NHP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, AAV1, AAV2, AAV3, AAV4, AAV5, AAV6, AAV7, AAV8, AAV9, AAV8 bp, AAV7M8 and AAVAnc80, variants of any of the known or mentioned AAVs or AAVs yet to be discovered or variants or mixtures thereof. See, e.g., WO 2005/033321, which is incorporated herein by reference. In another embodiment, the AAV capsid is an AAV8 bp capsid, which preferentially targets bipolar cells. See, WO 2014/024282, which is incorporated herein by reference. In another embodiment, the AAV capsid is an AAV7m8 capsid, which has shown preferential delivery to the outer retina. See, Dalkara et al, In Vivo-Directed Evolution of a New Adeno-Associated Virus for Therapeutic Outer Retinal Gene Delivery from the Vitreous, Sci Transl Med 5, 189ra76 (2013), which is incorporated herein by reference. In one embodiment, the AAV capsid is an AAV8 capsid. In another embodiment, the AAV capsid an AAV9 capsid. In another embodiment, the AAV capsid an AAV5 capsid.

In one embodiment, it is desirable to utilize an AAV capsid which shows tropism for the desired target cell, e.g., photoreceptors, RPE or other ocular cells. In one embodiment, the AAV capsid is a tyrosine capsid-mutant in which certain surface exposed tyrosine residues are substituted with phenylalanine (F). Such AAV variants are described, e.g., in Mowat et al, Tyrosine capsid-mutant AAV vectors for gene delivery to the canine retina from a subretinal or intravitreal approach, Gene Therapy 21, 96-105 (January 2014), which is incorporated herein by reference. In one embodiment the capsid is an AAV8 capsid with a Y733F mutation. In another embodiment, the capsid is an AAV8 capsid with Y447F, Y733F and T494V mutations (also called “AAV8(C&G+T494V)” and “rep2-cap8(Y447F+733F+T494V)”), as described by Kay et al, Targeting Photoreceptors via Intravitreal Delivery Using Novel, Capsid-Mutated AAV Vectors, PLoS One. 2013; 8(4): e62097. Published online 2013 Apr. 26, which is incorporated herein by reference. The coding sequence for a helper plasmid encoding rep2-cap8(Y447F+733F+T494V) is shown in SEQ ID NO: 9. The amino acid sequence for the AAV8(Y447F+733F+T494V) capsid is shown in SEQ ID NO: 10.

As used herein, relating to AAV, the term variant means any AAV sequence which is derived from a known AAV sequence, including those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3). In one embodiment, the AAV capsid shares at least 95% identity with the AAV8 vp3. In another embodiment, a self-complementary AAV is used.

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, Va.). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

As used herein, “artificial AAV” means, without limitation, an AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid.

For packaging an expression cassette or rAAV genome or production plasmid into virions, the ITRs are the only AAV components required in cis in the same construct as the transgene. In one embodiment, the coding sequences for the replication (rep) and/or capsid (cap) are removed from the AAV genome and supplied in trans or by a packaging cell line in order to generate the AAV vector. For example, as described above, a pseudotyped AAV may contain ITRs from a source which differs from the source of the AAV capsid. In one embodiment, AAV2/5 and AAV2/8 are exemplary pseudotyped vectors.

“Self-complementary AAV” refers a plasmid or vector having an expression cassette in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety. In one embodiment, the AAV is a self-complementary AAV2/8. See, e.g., Buie et al, Self-complementary AAV Virus (scAAV) Safe and Long-term Gene Transfer in the Trabecular Meshwork of Living Rats and Monkeys, Invest Ophthalmol Vis Sci. 2010 January; 51(1): 236-248, and Ryals et al, Quantifying transduction efficiencies of unmodified and tyrosine capsid mutant AAV vectors in vitro using two ocular cell lines, Mol Vis. 2011 Apr. 29; 17:1090-102, which are incorporated herein by reference. In one embodiment, the AAV is a self-complementary AAV2/8 having at least a Y733F mutation. See, Ku et al, Gene therapy using self-complementary Y733F capsid mutant AAV2/8 restores vision in a model of early onset Leber congenital amaurosis, Hum Mol Genet. 2011 Dec. 1; 20(23): 4569-4581, which is incorporated herein by reference. In another embodiment, the AAV is a self-complementary AAV2/8 having at least Y447F+733F+T494V mutations. See, Kay et al, 2013, cited herein.

In one embodiment, the vectors useful in compositions and methods described herein contain, at a minimum, sequences encoding a selected AAV serotype capsid, e.g., an AAV5 capsid, or a fragment thereof. In another embodiment, useful vectors contain, at a minimum, sequences encoding a selected AAV serotype rep protein, e.g., AAV5 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV5 origin.

Alternatively, vectors may be used in which the rep sequences are from an AAV serotype which differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199, which is incorporated by reference herein.

A suitable recombinant adeno-associated virus (AAV) is generated by culturing a host cell which contains a nucleic acid sequence encoding an adeno-associated virus (AAV) serotype capsid protein, or fragment thereof, as defined herein; a functional rep gene; a minigene composed of, at a minimum, AAV inverted terminal repeats (ITRs) and a NPHP5 nucleic acid sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Most suitably, such a stable host cell will contain the required component(s) under the control of an inducible promoter. However, the required component(s) may be under the control of a constitutive promoter. Examples of suitable inducible and constitutive promoters are provided herein, in the discussion below of regulatory elements suitable for use with the transgene, i.e., NPHP5. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing the rAAV of the invention may be delivered to the packaging host cell in the form of any genetic element which transfers the sequences carried thereon. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, 1993 J. Virol., 70:520-532 and U.S. Pat. No. 5,478,745, among others. These publications are incorporated by reference herein.

The minigene or vector genome is composed of, at a minimum, a NPHP5 nucleic acid sequence (the transgene), as described above, and its regulatory sequences, and 5′ and 3′ AAV inverted terminal repeats (ITRs). In one desirable embodiment, the ITRs of AAV serotype 2 are used. However, ITRs from other suitable serotypes may be selected. It is this minigene which is packaged into a capsid protein and delivered to a selected host cell.

The regulatory sequences include conventional control elements which are operably linked to the NPHP5 gene in a manner which permits its transcription, translation and/or expression in a cell transfected with the vector or infected with the virus produced by the invention. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, are known in the art and may be utilized.

The regulatory sequences useful in the constructs of the present invention may also contain an intron, desirably located between the promoter/enhancer sequence and the gene. One desirable intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV useful in the method of the invention is an internal ribosome entry site (IRES). An IRES sequence, or other suitable system, may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

The selection of the promoter to be employed in the rAAV may be made from among a wide number of constitutive or inducible promoters that can express the selected transgene in the desired an ocular cell. In another embodiment, the promoter is cell-specific. The term “cell-specific” means that the particular promoter selected for the recombinant vector can direct expression of the selected transgene in a particular ocular cell type. In one embodiment, the promoter is specific for expression of the transgene in photoreceptor cells. In another embodiment, the promoter is specific for expression in the rods and cones. In another embodiment, the promoter is specific for expression in the rods. In another embodiment, the promoter is specific for expression in the cones. In another embodiment, the promoter is specific for expression of the transgene in RPE cells. In another embodiment, the transgene is expressed in any of the above noted ocular cells.

The promoter may be derived from any species. In another embodiment, the promoter is the human G-protein-coupled receptor protein kinase 1 (GRK1) promoter (Genbank Accession number AY327580). In another embodiment, the promoter is a 292 nt fragment (positions 1793-2087) of the GRK1 promoter (SEQ ID NO: 5) (See also, Beltran et al, Gene Therapy 2010 17:1162-74, which is hereby incorporated by reference herein). In another preferred embodiment, the promoter is the human interphotoreceptor retinoid-binding protein proximal (IRBP) promoter. In one embodiment, the promoter is a 235 nt fragment of the hIRBP promoter (SEQ ID NO: 6).

In another embodiment, promoter is the native promoter for the gene to be expressed. In one embodiment, the promoter is the NPHP5 proximal promoter. Other promoters useful in the invention include, without limitation, the rod opsin promoter, the red-green opsin promoter, the blue opsin promoter, the cGMP-j-phosphodiesterase promoter, the mouse opsin promoter (Beltran et al 2010 cited above), the rhodopsin promoter (Mussolino et al, Gene Ther, July 2011, 18(7):637-45); the alpha-subunit of cone transducin (Morrissey et al, BMC Dev, Biol, Jan. 2011, 11:3); beta phosphodiesterase (PDE) promoter; the retinitis pigmentosa (RP 1) promoter (Nicord et al, J. Gene Med, December 2007, 9(12):1015-23); the NXNL2/NXNL1 promoter (Lambard et al, PLoS One, October 2010, 5(10):e13025), the RPE65 promoter; the retinal degeneration slow/peripherin 2 (Rds/perph2) promoter (Cai et al, Exp Eye Res. 2010 August; 91(2):186-94); and the VMD2 promoter (Kachi et al, Human Gene Therapy, 2009 (20:31-9)). Each of these documents is incorporated by reference herein. In another embodiment, the promoter is selected from human EF1α promoter, rhodopsin promoter, rhodopsin kinase, interphotoreceptor binding protein (IRBP), cone opsin promoters (red-green, blue), cone opsin upstream sequences containing the red-green cone locus control region, cone transducing, and transcription factor promoters (neural retina leucine zipper (Nrl) and photoreceptor-specific nuclear receptor Nr2e3, bZIP).

In another embodiment, the promoter is a ubiquitous or constitutive promoter. An example of a suitable promoter is a hybrid chicken β-actin (CBA) promoter with cytomegalovirus (CMV) enhancer elements. In another embodiment, the promoter is the CB7 promoter. Other suitable promoters include the human β-actin promoter, the human elongation factor-la promoter, the cytomegalovirus (CMV) promoter, the simian virus 40 promoter, and the herpes simplex virus thymidine kinase promoter. See, e.g., Damdindorj et al, (August 2014) A Comparative Analysis of Constitutive Promoters Located in Adeno-Associated Viral Vectors. PLoS ONE 9(8): e106472. Still other suitable promoters include viral promoters, constitutive promoters, regulatable promoters [see, e.g., WO 2011/126808 and WO 2013/04943]. Alternatively a promoter responsive to physiologic cues may be utilized in the expression cassette, rAAV genomes, vectors, plasmids and viruses described herein. In one embodiment, the promoter is of a small size, under 1000 bp, due to the size limitations of the AAV vector. In another embodiment, the promoter is under 400 bp. Other promoters may be selected by one of skill in the art.

Examples of constitutive promoters useful in the invention include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the chicken β-actin (CBA) promoter, the phosphoglycerol kinase (PGK) promoter, the EF1 promoter (Invitrogen), and the immediate early CMV enhancer coupled with the CBA promoter (Beltran et al, Gene Therapy 2010 cited above).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system and the rapamycin-inducible system. Other types of inducible promoters which may be useful in this context are those which are regulated by a specific physiological state, e.g., temperature, acute phase, a particular differentiation state of the cell, or in replicating cells only. Any type of inducible promoter which is tightly regulated and is specific for the particular target ocular cell type may be used.

In other embodiments, the cassette, vector, plasmid and virus constructs described herein contain other appropriate transcription initiation, termination, enhancer sequences, efficient RNA processing signals such as splicing and polyadenylation (polyA) signals; TATA sequences; sequences that stabilize cytoplasmic mRNA; sequences that enhance translation efficiency (i.e., Kozak consensus sequence); introns; sequences that enhance protein stability; and when desired, sequences that enhance secretion of the encoded product. The expression cassette or vector may contain none, one or more of any of the elements described herein. Examples of suitable polyA sequences include, e.g., SV40, bovine growth hormone (bGH), and TK polyA. Examples of suitable enhancers include, e.g., the CMV enhancer, the RSV enhancer, the alpha fetoprotein enhancer, the TTR minimal promoter/enhancer, LSP (TH-binding globulin promoter/alpha1-microglobulin/bikunin enhancer), amongst others.

Exemplary plasmids for use in producing the compositions described herein are provided. SEQ ID NO: 7 shows pTR-hIRBP-cNPHP5. SEQ ID NO: 8 shows Sc-hGRK1-cNPHP5. A human NPHP5 sequence, such as that shown in SEQ ID NO: 4 can be substituted for the canine sequences encoded therein.

Other enhancer sequences useful in the invention include the IRBP enhancer (Nicord 2007, cited above), immediate early cytomegalovirus enhancer, one derived from an immunoglobulin gene or SV40 enhancer, the cis-acting element identified in the mouse proximal promoter, etc.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989). Of course, not all vectors and expression control sequences will function equally well to express all of the transgenes of this invention. However, one of skill in the art may make a selection among these, and other, expression control sequences without departing from the scope of this invention.

An example of a suitable vector genome sequence containing the canine NPHP5 coding sequence is shown in SEQ ID NO: 11. Such sequence was used in the exemplary AAV2/5-hIRBP-cNPHP5 construct described in the examples herein. Another example of a suitable vector genome sequence, containing the canine NPHP5 coding sequence, is shown in SEQ ID NO: 12. Such sequence was used in the exemplary scAAV2/8-hGRK1-cNPHP5 virus and scAAV2/8mutC&G+T494V-hGRK1-cNPHP5 constructs described in the examples herein. Similar vector genomes in which the canine NPHP5 sequence is swapped with a human NPHP5 sequence are encompassed herein, e.g., SEQ ID NO: 13 and 14 respectively.

### E. PHARMACEUTICAL COMPOSITIONS AND ADMINISTRATION

The recombinant AAV containing the desired transgene and cell-specific promoter for use in the target ocular cells as detailed above is preferably assessed for contamination by conventional methods and then formulated into a pharmaceutical composition intended for subretinal injection. Such formulation involves the use of a pharmaceutically and/or physiologically acceptable vehicle, carrier, buffer, diluent and/or adjuvant, etc. particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20.

In one exemplary embodiment, the composition of the carrier or excipient contains 180 mM NaCl, 10 mM NaPi, pH7.3 with 0.0001%-0.01% Pluronic F68 (PF68). The exact composition of the saline component of the buffer ranges from 160 mM to 180 mM NaCl. Optionally, a different pH buffer (potentially HEPES, sodium bicarbonate, TRIS) is used in place of the buffer specifically described. Still alternatively, a buffer containing 0.9% NaCl is useful.

Optionally, the compositions of the invention may contain, in addition to the rAAV and/or variants and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The pharmaceutical compositions containing at least one replication-defective rAAV virus, as described herein, can be formulated with a physiologically acceptable carrier, diluent, excipient and/or adjuvant, for use in gene transfer and gene therapy applications. In the case of AAV viral vectors, quantification of the genome copies (“GC”), vector genomes (“VG”), or virus particles may be used as the measure of the dose contained in the formulation or suspension. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal). In another method the effective dose of a recombinant adeno-associated virus carrying a nucleic acid sequence encoding the optimized NPHP5 transgene is measured as described in S. K. McLaughlin et al, 1988 J. Virol., 62:1963, which is incorporated by reference in its entirety. In another method, the titer is determined using droplet digital PCR (ddPCR). See, Lock as described in, e.g., M. Lock et al, Hu Gene Therapy Methods, 2014 April; 25(2):115-25. doi: 10.1089/hgtb.2013.131. Epub 2014 Feb. 14, which is incorporated herein by reference.

As used herein, the term “dosage” can refer to the total dosage delivered to the subject in the course of treatment, or the amount delivered in a single unit (or multiple unit or split dosage) administration. The pharmaceutical virus compositions can be formulated in dosage units to contain an amount of replication-defective virus carrying the nucleic acid sequences encoding NPHP5 as described herein that is in the range of about 1.0×10⁸ GC to about 1.0×10¹⁵ GC including all integers or fractional amounts within the range. In one embodiment, the compositions are formulated to contain at least 1×10⁸, 2×10⁸, 3×10⁸, 4×10⁸, 5×10⁸, 6×10⁸, 7×10⁸, 8×10⁸, or 9×10⁸ GC per dose including all integers or fractional amounts within the range. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³ 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, Or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵ 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 to about 1000 microliters, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 70 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In one embodiment, the viral constructs may be delivered in doses of from at least 1×10⁷ to about least 1×10¹¹ GCs in volumes of about 11 μL to about 3 μL for small animal subjects, such as mice. For larger veterinary subjects having eyes about the same size as human eyes, the larger human dosages and volumes stated above are useful. See, e.g., Diehl et al, J. Applied Toxicology, 21:15-23 (2001) for a discussion of good practices for administration of substances to various veterinary animals. This document is incorporated herein by reference.

It is desirable that the lowest effective concentration of virus or other delivery vehicle be utilized in order to reduce the risk of undesirable effects, such as toxicity, retinal dysplasia and detachment. Still other dosages in these ranges may be selected by the attending physician, taking into account the physical state of the subject, preferably human, being treated, the age of the subject, the particular ocular disorder and the degree to which the disorder, if progressive, has developed.

Yet another aspect described herein is a method for treating, retarding or halting progression of blindness in a mammalian subject having, or at risk of developing, NPHP5-LCA. In one embodiment, a rAAV carrying the NPHP5 coding sequence, preferably suspended in a physiologically compatible carrier, diluent, excipient and/or adjuvant, may be administered to a desired subject including a human subject. This method comprises administering to a subject in need thereof any of the nucleic acid sequences, expression cassettes, rAAV genomes, plasmids, vectors or rAAV vectors or compositions containing them. In one embodiment, the composition is delivered subretinally. In another embodiment, the composition is delivered intravitreally. In still another embodiment, the composition is delivered using a combination of administrative routes suitable for treatment of ocular diseases, and may also involve administration via the palpebral vein or other intravenous or conventional administration routes.

Yet another aspect described herein is a method for treating, retarding or halting progression of blindness in a mammalian subject having, or at risk of developing, LCA ciliopathy. In one embodiment, a rAAV carrying the NPHP5 coding sequence, preferably suspended in a physiologically compatible carrier, diluent, excipient and/or adjuvant, may be administered to a desired subject including a human subject. This method comprises administering to a subject in need thereof any of the nucleic acid sequences, expression cassettes, rAAV genomes, plasmids, vectors or rAAV vectors or compositions containing them. In one embodiment, the composition is delivered subretinally. In another embodiment, the composition is delivered intravitreally. In still another embodiment, the composition is delivered using a combination of administrative routes suitable for treatment of ocular diseases, and may also involve administration via the palpebral vein or other intravenous or conventional administration routes.

Furthermore, in certain embodiments of the invention it is desirable to perform non-invasive retinal imaging and functional studies to identify areas of retained photoreceptors to be targeted for therapy. In these embodiments, clinical diagnostic tests are employed to determine the precise location(s) for one or more subretinal injection(s). These tests may include electroretinography (ERG), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc. These, and other desirable tests, are known in the art.

In view of the imaging and functional studies, in some embodiments of the invention one or more injections are performed in the same eye in order to target different areas of retained photoreceptors. The volume and viral titer of each injection is determined individually, as further described herein, and may be the same or different from other injections performed in the same, or contralateral, eye. In another embodiment, a single, larger volume injection is made in order to treat the entire eye. In one embodiment, the volume and concentration of the rAAV composition is selected so that only the region of damaged photoreceptors is impacted. In another embodiment, the volume and/or concentration of the rAAV composition is a greater amount, in order reach larger portions of the eye, including non-damaged photoreceptors.

The composition may be delivered in a volume of from about 50 μL to about 1 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. An effective concentration of a recombinant adeno-associated virus carrying a nucleic acid sequence encoding the desired transgene under the control of the cell-specific promoter sequence desirably ranges between about 10⁸ and 10¹³ vector genomes per milliliter (vg/mL). The rAAV infectious units are measured as described in S. K. McLaughlin et al, 1988 J. Virol., 62:1963. In one embodiment, the concentration is from about 1.5×10⁹ vg/mL to about 1.5×10¹² vg/mL. In another, from about 1.5×10⁹ vg/mL to about 1.5×10¹¹ vg/mL. In one embodiment, the effective concentration is about 1.5×10¹⁰ vg/mL. In another embodiment, the effective concentration is about 1.5×10¹¹ vg/mL. In another embodiment, the effective concentration is about 2.8×10¹¹ vg/mL. In yet another embodiment, the effective concentration is about 1.5×10¹² vg/mL. In another embodiment, the effective concentration is about 1.5×10¹³ vg/mL. It is desirable that the lowest effective concentration of virus be utilized in order to reduce the risk of undesirable effects, such as toxicity, retinal dysplasia and detachment. Still other dosages in these ranges may be selected by the attending physician, taking into account the physical state of the subject, preferably human, being treated, the age of the subject, the particular ocular disorder and the degree to which the disorder, if progressive, has developed.

### F. METHODS OF TREATMENT/PROPHYLAXIS

The invention provides various methods of preventing, treating, arresting progression of or ameliorating the above-described ocular diseases and retinal changes associated therewith. Generally, the methods include administering to a mammalian subject in need thereof, an effective amount of a composition comprising a recombinant adeno-associated virus (AAV) carrying a nucleic acid sequence encoding a normal NPHP5 protein, or fragment thereof, under the control of regulatory sequences which express the product of the gene in the subject's ocular cells, and a pharmaceutically acceptable carrier.

In a particular embodiment, the invention provides a method of preventing, arresting progression of or ameliorating vision loss associated with Leber congenital amaurosis in the subject. Vision loss associated with LCA refers to any decrease in peripheral vision, central (reading) vision, night vision, day vision, loss of color perception, loss of contrast sensitivity, or reduction in visual acuity. Other vision problems that may be treated using the described methods include photophobia and nystagmus.

In another embodiment, the invention provides a method to prevent, or arrest photoreceptor function loss, or increase photoreceptor function in the subject.

Photoreceptor function may be assessed using the functional studies described above and in the examples below, e.g., ERG or perimetry, which are conventional in the art. As used herein “photoreceptor function loss” means a decrease in photoreceptor function as compared to a normal, non-diseased eye or the same eye at an earlier time point. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient.

In another aspect, the invention provides method of improving photoreceptor structure in the subject. As used herein “improving photoreceptor structure” refers (in the region of the retina that is treated) to one or more of an increase or decrease in outer nuclear layer (ONL) thickness, or arresting progression of ONL thickening or thinning, across the entire retina, in the central retina, or the periphery; increase or decrease in outer plexiform layer (OPL) thickness, or arresting progression of OPL thickening or thinning, across the entire retina, in the central retina, or the periphery; decrease in rod and cone inner segment (IS) shortening; decrease in shortening and loss of outer segments (OS); decrease in bipolar cell dendrite retraction, or an increase in bipolar cell dendrite length or amount; and reversal of opsin mislocalization.

In another aspect, the invention provides a method of preventing NPHP5-LCA in a subject at risk of developing said disease. Subjects at risk of developing NPHP5 include those with a family history of NPHP5-LCA, those with a family history of Senior Loken syndrome, and those with one or more confirmed mutations in the NPHP5 gene.

For each of the described methods, the treatment may be used to prevent the occurrence of retinal damage or to rescue eyes having mild or advanced disease. As used herein, the term “rescue” means to prevent progression of the disease to total blindness, prevent spread of damage to uninjured photoreceptor cells or to improve damage in injured photoreceptor cells. Thus, in one embodiment, the composition is administered before disease onset. In another embodiment, the composition is administered after the initiation of opsin mislocalization. In another embodiment, the composition is administered prior to the initiation of photoreceptor loss. In another embodiment, the composition is administered after initiation of photoreceptor loss. In yet another embodiment, the composition is administered when less than 90% of the photoreceptors are functioning or remaining, as compared to a non-diseased eye. In another embodiment, the composition is administered when less than 80% of the photoreceptors are functioning or remaining. In another embodiment, the composition is administered when less than 70% of the photoreceptors are functioning or remaining. In another embodiment, the composition is administered when less than 60% of the photoreceptors are functioning or remaining. In another embodiment, the composition is administered when less than 50% of the photoreceptors are functioning or remaining. In another embodiment, the composition is administered when less than 40% of the photoreceptors are functioning or remaining. In another embodiment, the composition is administered when less than 30% of the photoreceptors are functioning or remaining. In another embodiment, the composition is administered when less than 20% of the photoreceptors are functioning or remaining. In another embodiment, the composition is administered when less than 10% of the photoreceptors are functioning or remaining. In one embodiment, the composition is administered only to one or more regions of the eye, e.g., those which have retained photoreceptors. In another embodiment, the composition is administered to the entire eye.

In another embodiment, a method of treating or preventing NPHP5-LCA in a subject in need thereof is provided. The method includes identifying a subject having, or at risk of developing, NPHP5-LCA; performing genotypic analysis and identifying at least one mutation in the NPHP5 gene; performing non-invasive retinal imaging and functional studies and identifying areas of retained photoreceptors to be targeted for therapy; and administering to the subject an effective concentration of a composition, whereby NPHP5-LCA is prevented, arrested or ameliorated. The composition includes a recombinant virus carrying a nucleic acid sequence encoding a normal photoreceptor cell-specific gene under the control of a promoter sequence which expresses the product of the gene in the photoreceptor cells, and a pharmaceutically acceptable carrier. Genotypic analysis is routine in the art and may include the use of PCR to identify one or more mutations in the nucleic acid sequence of the NPHP5 gene. See, e.g., Meindl et al, Nat Gen, May 1996, 13:35, Vervoort, R. et al, 2000. Nat Genet 25(4): 462-466 (cited above); and Vervoort, R. and Wright, A. F. 2002. Human Mutation 19: 486-500, each of which is incorporated herein by reference.

In another embodiment, any of the above methods are performed utilizing a composition comprising a recombinant AAV2/5 pseudotyped adeno-associated virus, carrying a nucleic acid sequence encoding a normal NPHP5 protein, or fragment thereof, under the control of an IRBP promoter which directs expression of the product of the gene in the photoreceptor cells of the subject, formulated with a carrier and additional components suitable for subretinal injection.

In another embodiment, any of the above methods are performed utilizing a composition comprising a recombinant scAAV2/8 pseudotyped adeno-associated virus with a single capsid tyrosine modification (Y733F), carrying a nucleic acid sequence encoding a normal NPHP5 protein, or fragment thereof, under the control of a GRK1 promoter which directs expression of the product of the gene in the photoreceptor cells of the subject, formulated with a carrier and additional components suitable for subretinal injection.

In another embodiment of the invention, the method includes performing functional and imaging studies to determine the efficacy of the treatment. These studies include ERG and in vivo retinal imaging, as described in the examples below. In addition visual field studies, perimetry and microperimetry, mobility testing, visual acuity, color vision testing may be performed.

In yet another embodiment of the invention, any of the above described methods is performed in combination with another, or secondary, therapy. The therapy may be any now known, or as yet unknown, therapy which helps prevent, arrest or ameliorate NPHP5-LCA or any of the above-described effects associated therewith. The secondary therapy can be administered before, concurrent with, or after administration of the rAAV described above. In one embodiment, the secondary therapy is a neuroprotective therapy.

In one embodiment, the method is performed more than once. Such subsequent injections can occur with the same vector construct or a different one, such as that utilizing a different AAV capsid vector. In one embodiment, the subsequent injection occurs days, weeks, months or one or more years after the first treatment.

As is demonstrated in the examples below, an exemplary cNPHP5 was employed in in vivo experiments to provide evidence of the utility and efficacy of the methods and compositions of this invention. The examples demonstrated restoration of retinal function by the method of this invention in a large animal model of a human LCA. The use of the exemplary vector demonstrated in the experiments that the defect in the NPHP5 mutant dogs could be corrected by gene delivery. Retinal function was improved in this large animal model of blindness. This data allow one of skill in the art to readily anticipate that this method may be similarly used in treatment of NPHP5-LCA and other types of LCA-ciliopathy in other subjects, including humans.

### G. EXAMPLES

### Example 1: Materials and Methods

To determine if canine NPHP5 gene augmentation with either AAV2/5-IRBP or AAV2/8 (Y733F)-scGRK1 rescues retinal degeneration in mutant NPHP5 dogs when delivered by subretinal injection at 5.7 weeks of age, animals were treated as follows:

| Dog | Genotype | Sex | Age at injection | Right Eye (OD) | Left Eye (OS) |
|---|---|---|---|---|---|
| AS21-7 | Crd2(A) | F | 5.7 weeks | Non-injected | AAV2/5 |
| | −/− | | | | IRBP-cNPHP5 |
| | Crd1(C) | | | | 1.5E+12 vg/ml |
| | −/+ | | | | 70 μl |
| AS2-389 | Crd2(A) | F | 5.7 weeks | Non-injected | AAV2/8(Y733F) |
| | −/− | | | | scGRK1-cNPHP5 |
| | | | | | 1.5E+11 vg/ml |
| | | | | | 70 μl |
| AS2-391 | Crd2(A) | F | 5.7 weeks | Non-injected | AAV2/8(Y733F) |
| | −/− | | | | scGRK1-cNPHP5 |
| | | | | | 1.5E+12 vg/ml |
| | | | | | 70 μl |

On date of injection, pupils were dilated (3× at 30 min interval) with Tropicamide/Phenylephrine/Atropine. Subretinal (SR) injection aiming for the Area Centralis was performed under (propofol induction) isoflurane gas anesthesia. The injected viral preparation (˜70 μl) contained the test vector listed in the table above and a small amount of an AAV2/5 carrying the reporter gene GFP to facilitate detection at later time points of the treated area by non-invasive retinal imaging (scanning confocal laser ophthalmoscopy, autofluorescence mode).

Eye exams were performed pre-injection, 24 hrs PI and on a weekly basis for 8 weeks, then monthly. At the following time points assessment of retinal function by electroretinography (ERG) was performed in each eye: at approx. 13, 20, 32, 49, 65, 79, 99, and 125 weeks of age. Retinal structure and outer nuclear layer (ONL) thickness was assessed by cSLO/OCT non-invasive retinal imaging in each eye at approx. 14, 33, 51, 66, 79, 97, and 125 weeks of age.

Results:

Because of its high transduction efficiency for RPGR mutant rods and cones, the vector construct AAV2/5-hIRBP- used in a different project for a different disease (Gene augmentation therapy for RPGR-X-linked retinitis pigmentosa)—was tested initially, in NPHP5 mutant dogs that were treated with the wild type canine NPHP5 cDNA. NPHP5 mutant dogs were initially injected subretinally with 70 μl at a 1.5×10¹¹ vg/ml titer at 7.5 wks with AAV2/5-hIRBP-cNPHP5. Treatment did not rescue function at any time point up to 33 wks (data not shown). Treatment at 5.7 wks of age with a 10-fold increase in titer to 1.5×10¹² vg/ml had a positive but modest effect on improving rod (FIG. 1, Left column) and cone (FIG. 2, Left column) ERG function with time. Maximal ERG recovery was reached by 79 weeks and was still stable at 125 weeks of age. Thus a positive rescue effect on ERG function was observed for >2 years. Treatment also had positive effect on preservation of retinal vasculature and outer nuclear layer (ONL) thickness in the treated area of the injected eye, while ongoing degeneration occurred in surrounding untreated areas as well as in the contralateral uninjected eye (FIGS. 3-8, FIG. 21, top row).

To increase the transduction efficiency, the hGRK1 promoter was used as this promoter is highly effective in other canine retinal degenerative diseases treated by gene augmentation. As well, a self-complementary AAV2/8 vector was used to speed up transgene expression as it bypasses the need to convert single-stranded DNA genome into double-stranded DNA prior to expression, and has a single capsid tyrosine modification (Y733F) that increases nuclear targeting. Treatment at 5.7 wk with this vector [1.5×10¹¹ vg/ml titer; 70 μl vol; scAAV2/8(Y733F)-hGRK1-cNPHP5] resulted in modest functional recovery that is comparable to the AAV2/5-hIRBP-cNPHP5 vector used at the higher dose (FIGS. 1 and 2, compare middle column to left column). However, when a 1.5×10¹² vg/ml titer of the scAAV2/8(Y733F)-hGRK1-cNPHP5 with tyrosine capsid mutation vector was used, there was remarkable recovery of cone function, and preservation of cone/rod ERG and vision for the 2 year observation time period (FIGS. 1 and 2, right column). Similarly, improved preservation of the retina and ONL thickness was observed with the scAAV2/8(Y733F)-hGRK1-cNPHP5 vector construct when used at a titer of 1.5×10¹² vg/ml rather than 1.5×10¹¹ vg/ml (FIGS. 9-20, FIG. 21, middle and lower rows).

No clinical signs of ocular/retinal toxicity were observed in any of the eyes treated with the vectors listed above throughout the in life study duration.

### Example 2

An experiment was designed to determine if half log higher titer (4.74×10¹² vg/ml) of AAV2/8 (Y733F)-scGRK1-cNPHP5 provides stable ERG rescue (Dog AS2-407); to the test same construct but with human NPHP5 transgene instead (Dog AS2-405); and to test the canine NPHP5 transgene in a new capsid variant: AAV2/8mut C&G+T494V-scGRK1-cNPHP5 (aka, with Y447F+733F+T494V mutations)(dog AS2-406). All viral vector constructs were delivered at early stage of disease (5.7 wks of age).

Animals were treated as follows:

| Dog | Genotype | Sex | Age at injection | Right Eye (OD) | Left Eye (OS) |
|---|---|---|---|---|---|
| AS2-407 | crd2 A | F | 5.7 wks | Not injected | sc-AAV2/8(Y733F)- |
| | | | | | GRK1-cNPHP5 |
| | | | | | 4.74 × 10¹² vg/ml |
| | | | | | 70 ul SR |
| AS2-405 | crd2 A | M | 5.7 wks | sc-AAV2/8(Y733F)- | sc-AAV2/8(Y733F)- |
| | | | | GRK1-hNPHP5 | GRK1-hNPHP5 |
| | | | | 1.5 × 10¹² vg/ml | 4.74 × 10¹² vg/ml |
| | | | | 70 ul SR | 70 ul SR |
| AS2-406 | crd2 A | F | 5.7 wks | sc-AAV2/8mutC&G+ | sc-AAV2/8mutC&G+ |
| | | | | T494V-GRK1-cNPHP5 | T494V-GRK1-cNPHP5 |
| | | | | 1.5 × 10¹² vg/ml | 4.74 × 10¹² vg/ml |
| | | | | 70 ul SR | 70 ul SR |

On date of injection, pupils were dilated (3× at 30 min interval) with Tropicamide/Phenylephrine/Atropine. Subretinal (SR) injection aiming for the Area Centralis was performed under (propofol induction) isoflurane gas anesthesia. The injected viral preparation (˜70 μl) contained the test vector listed in the table above and a small amount of an AAV2/5 carrying the reporter gene GFP to facilitate detection at later time points of the treated area by non-invasive retinal imaging (scanning confocal laser ophthalmoscopy, autofluorescence mode).

Eye exams were performed pre-injection, 24 hrs (PI) and on a weekly basis for 8 weeks, then monthly. At the following time points assessment of retinal function by electroretinography (ERG) was performed in each eye: at approx. 13, 20, and 31 weeks of age.

### Results:

No clinical signs of ocular/retinal toxicity were observed in any of the eyes treated with the vectors listed above throughout the in life study duration.

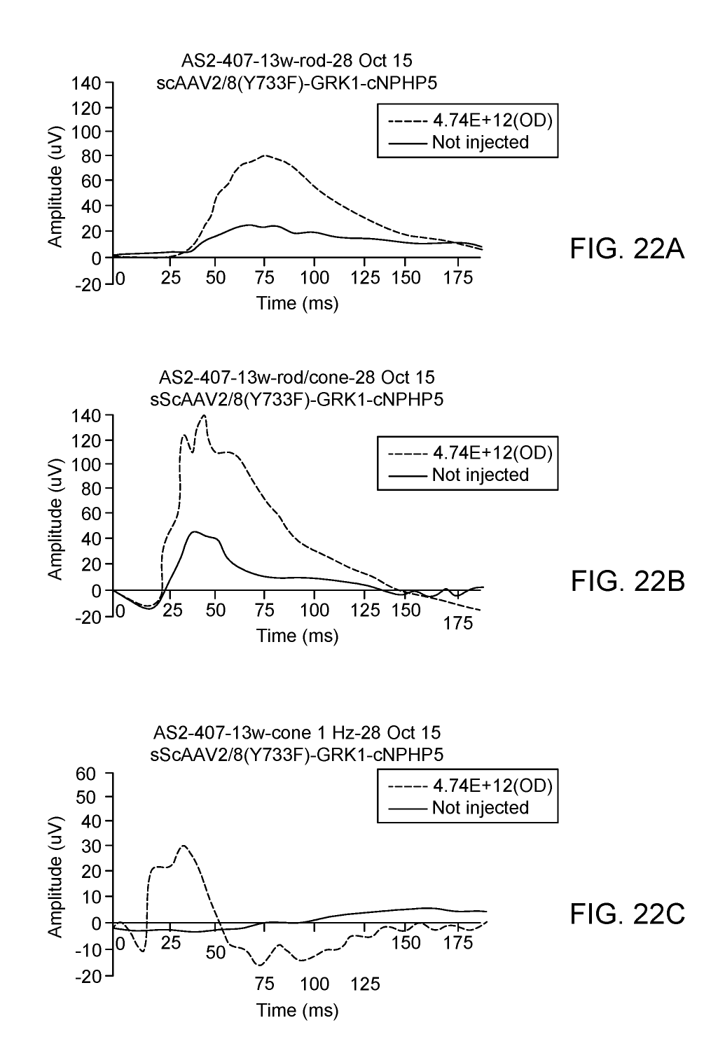

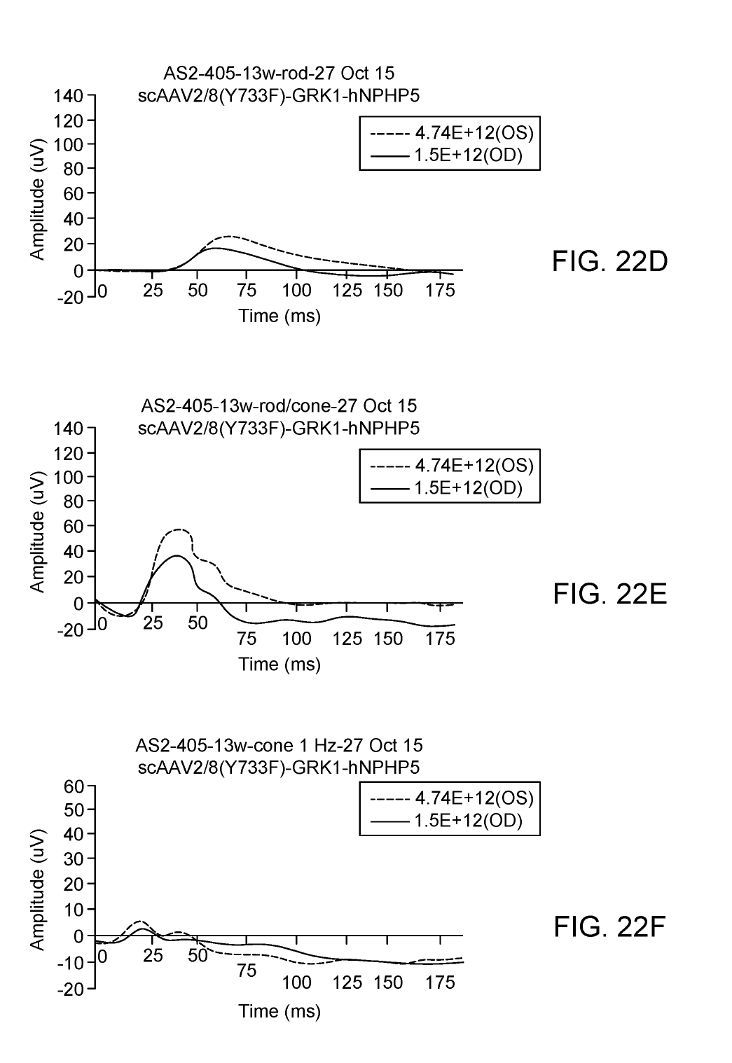

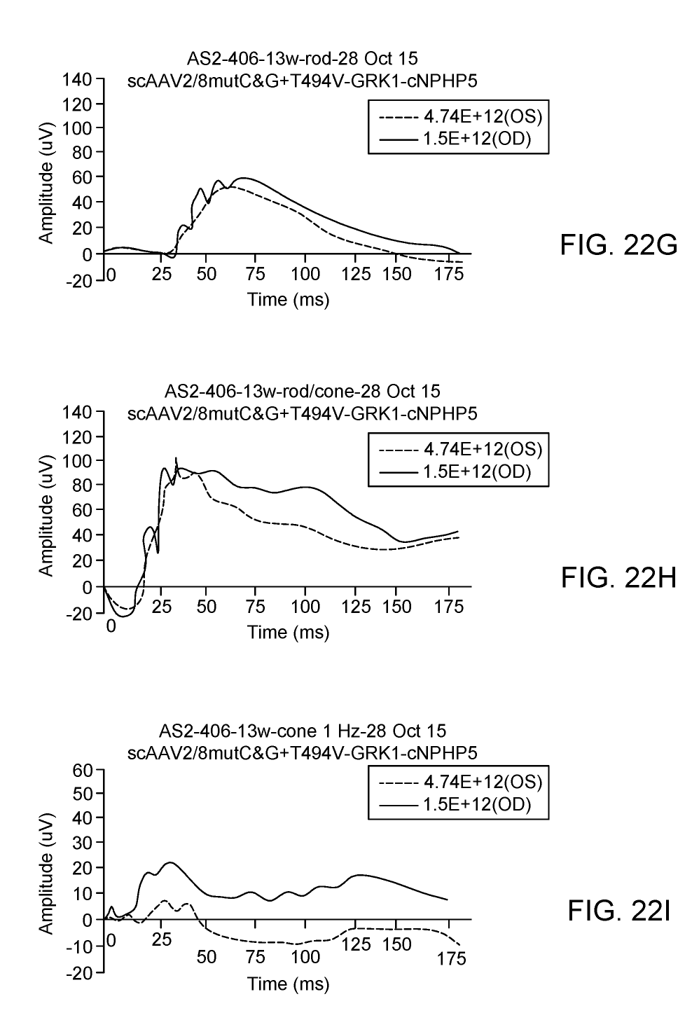

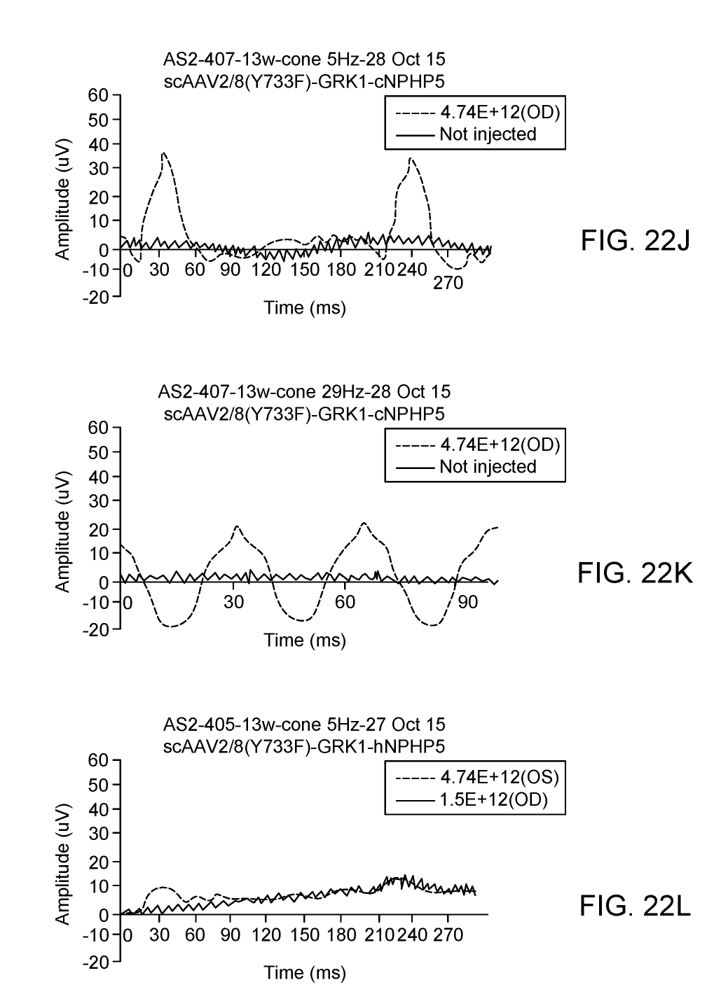

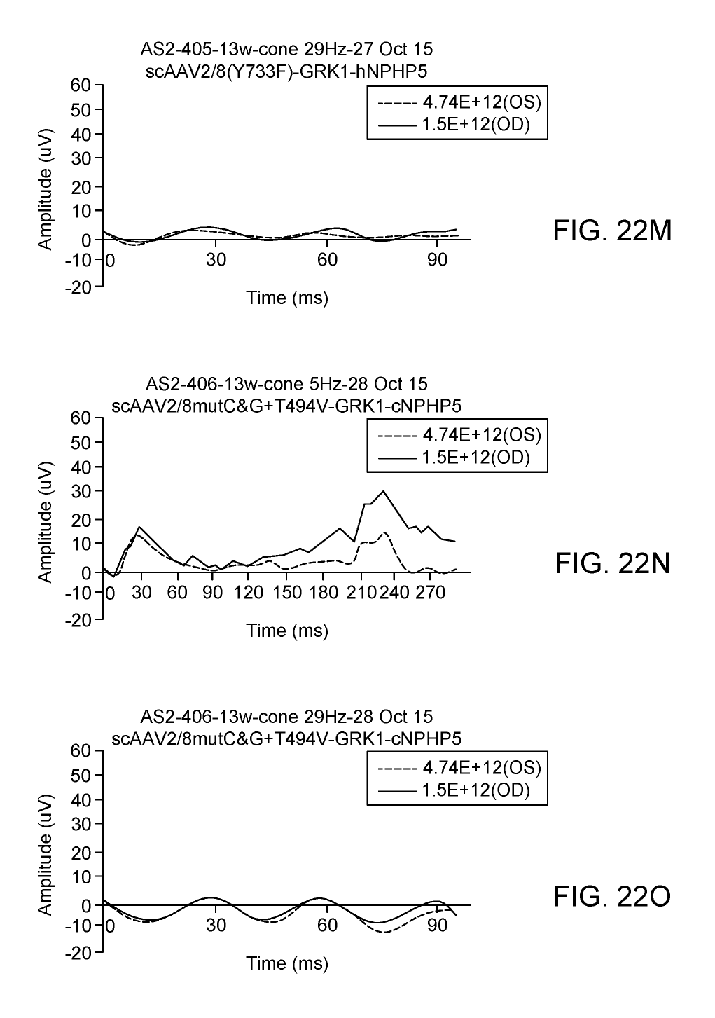

The scAAV2/8(Y733F)-GRK1-cNPHP5 vector construct delivered by subretinal injection at 4.74×10¹² vg/ml titer (70 ul volume) at the onset of disease (5.7 weeks of age) provided at 13 weeks improved rod and cone ERG function (FIG. 22, Left column) that was better than that achieved at the same age with a lower titer of 1.5×10¹² vg/ml (see Example 1).

With the scAAV2/8(Y733F)-GRK1-cNPHP5 vector construct that carried the human NPHP5 transgene, only very modest rod and cone ERG rescue (FIG. 22, center column) was achieved in the single treated NPHP5 mutant dog at 13 weeks of age with 1.5×10¹² and 4.74×10¹² vg/ml titers.

Finally, with the scAAV2/8mutC&G+T494V-GRK1-cNPHP5 vector construct rescue of both rod and cone ERG function (FIG. 22, Right column) was achieved at 13 weeks of age following subretinal injection with both 1.5×10¹² and 4.74×10¹² vg/ml titers.

For the 3 vectors described above ERG results were stable until end of the study at 31 weeks of age (data not shown).

### Example 3

An experiment was designed to further evaluate the canine NPHP5 transgene in the capsid variant scAAV2/8mut C&G+T494V at a later age. The scAAV2/8mut C&G+T494V-GRK1-cNPHP5 vector construct was delivered by subretinal injection in NPHP5 mutant dogs after the onset of retinal degeneration (at 8.6 wks of age).

Animals were treated as follows:

| | | | | Age at | | |
|---|---|---|---|---|---|---|
| Dog | Genotype | Sex | DOB | injection | Right Eye (OD) | Left Eye (OS) |
| WM27 | crd2 A | M | Oct. 17, 2015 | 8.6 wks | Not injected | scAAV2/8mut |
| | | | | | | C&G+T494V− |
| | | | | | | GRK1−cNPHP5 |
| | | | | | | 4.74E+12 vg/ml |
| | | | | | | 100 μl SR |
| WM28 | crd2 A | M | Oct. 17, 2015 | 8.6 wks | Not injected | scAAV2/8mut |
| | | | | | | C&G+T494V− |
| | | | | | | GRK1−cNPHP5 |
| | | | | | | 4.74E+12 vg/ml |
| | | | | | | 100 μl SR |

On date of injection, pupils were dilated (3× at 30 min interval) with Tropicamide/Phenylephrine/Atropine. Subretinal (SR) injection aiming for the Area Centralis was performed under (propofol induction) isoflurane gas anesthesia. The injected viral preparation (˜100 μl) contained the test vector listed in the table above and a small amount of an AAV2/5 carrying the reporter gene GFP to facilitate detection at later time points of the treated area by non-invasive retinal imaging (scanning confocal laser ophthalmoscopy, autofluorescence mode).

Eye exams were performed pre-injection, 24 hrs PI and on a weekly basis for 8 weeks, then monthly. At the following time points assessment of retinal function by electroretinography (ERG) was performed in each eye: at approx. 33, 53, and 67 weeks of age. Retinal structure and outer nuclear layer (ONL) thickness was assessed by cSLO/OCT non-invasive retinal imaging in each eye at 7 weeks of age (pre-injection time point) and after injection at 20, 49, and 65 weeks of age.

Results: No clinical signs of ocular/retinal toxicity were observed in any of the eyes treated with the vector listed above throughout the in life study duration.

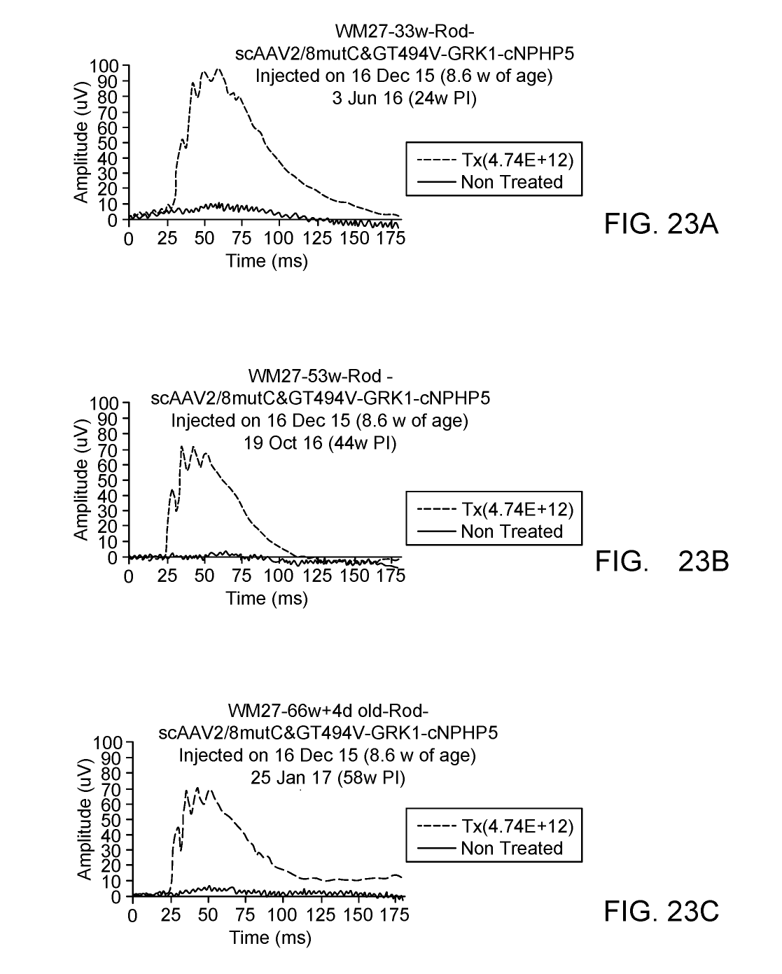

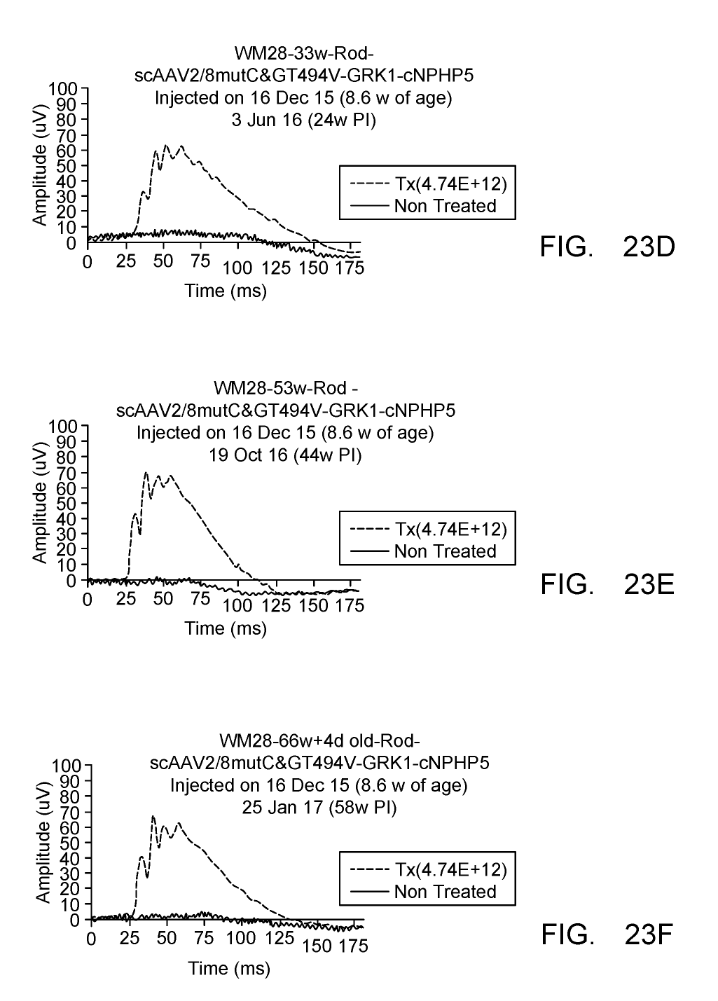

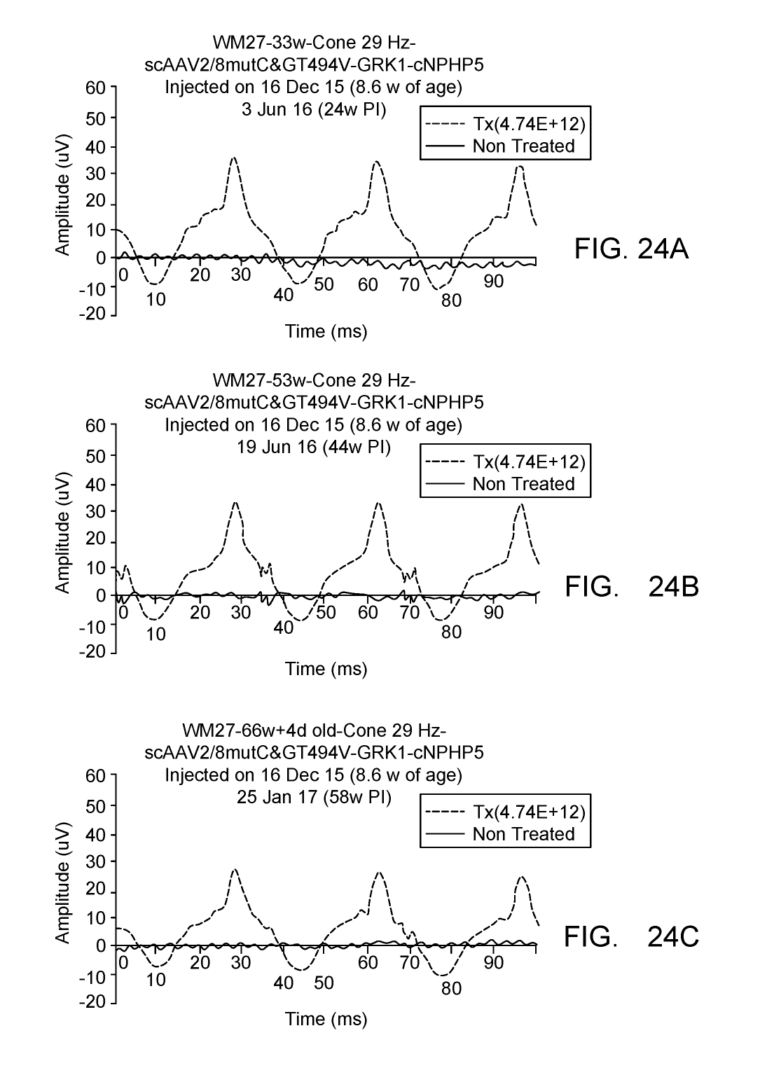

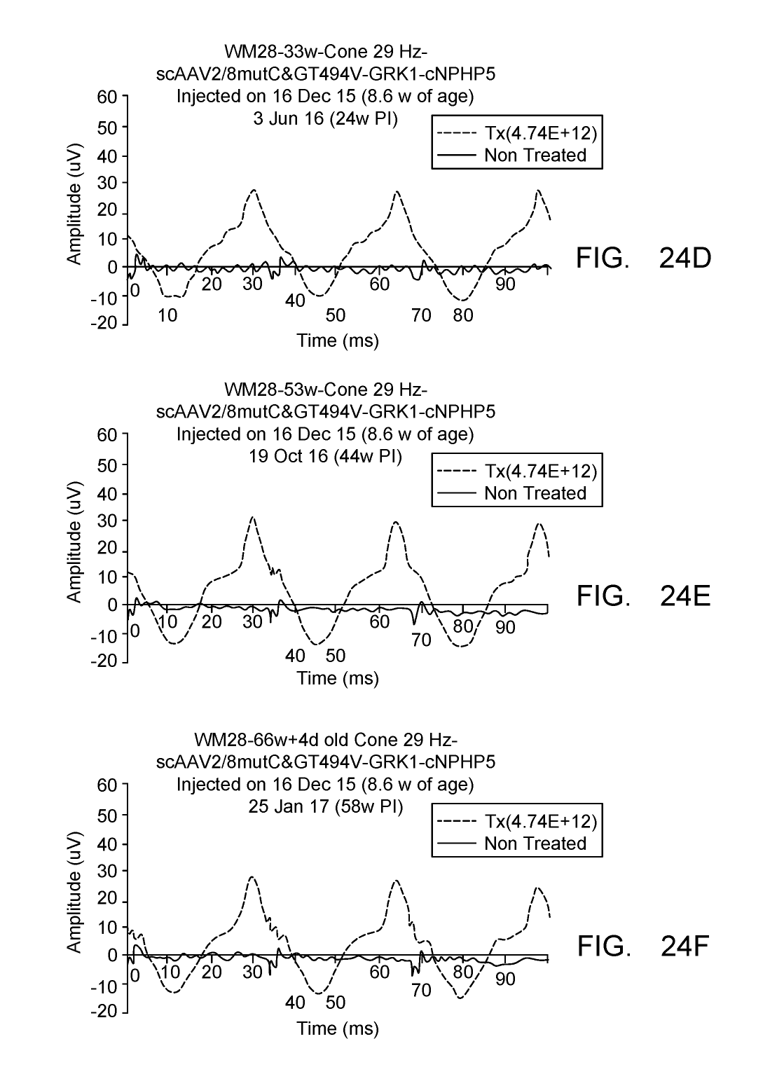

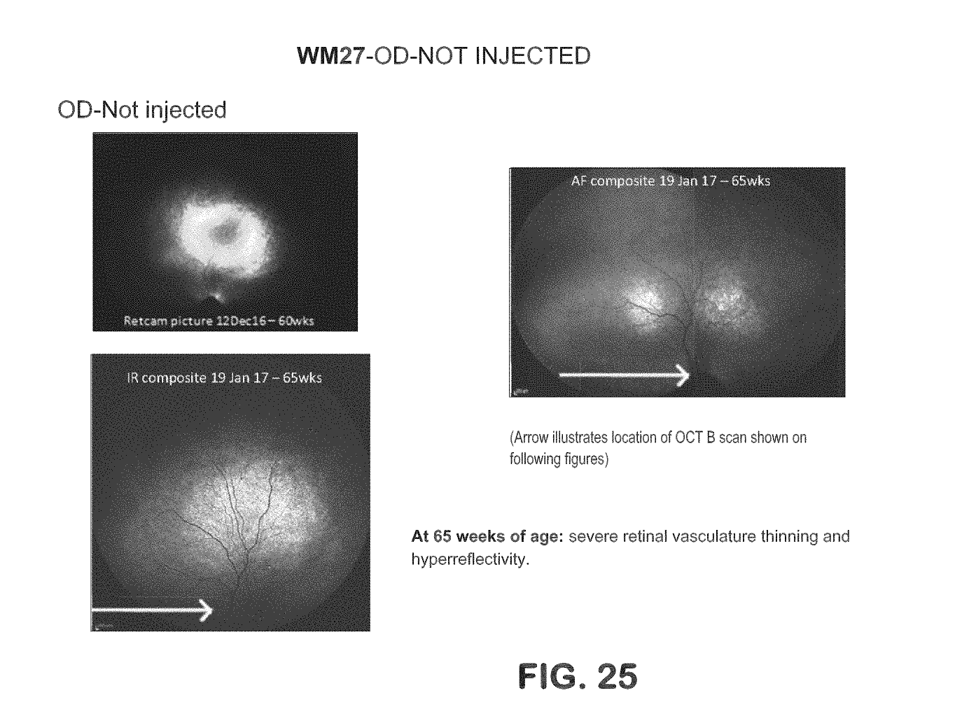

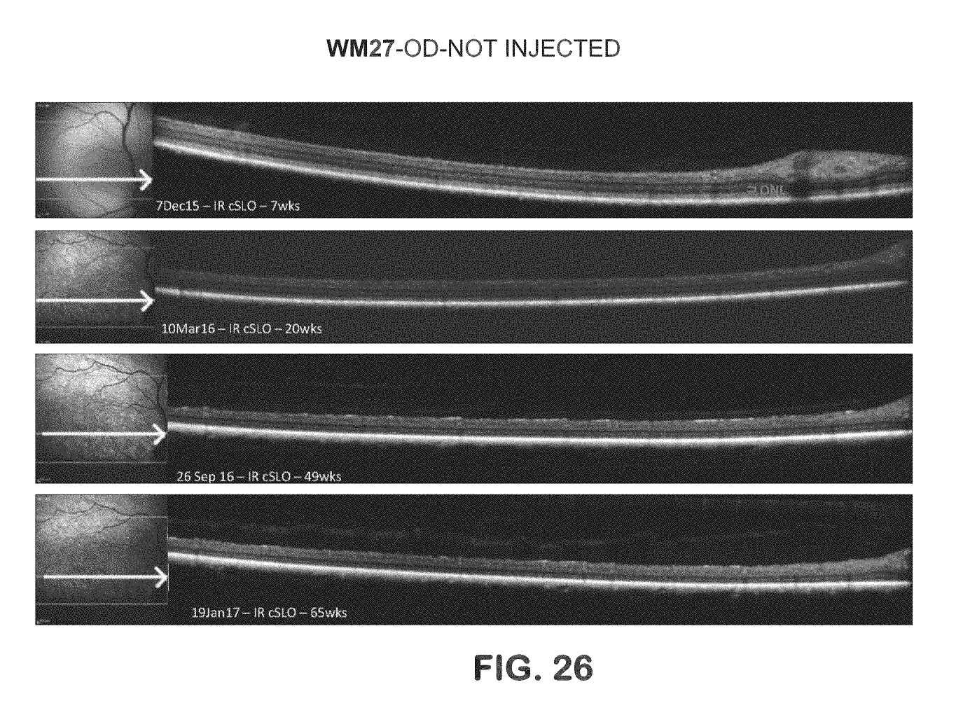

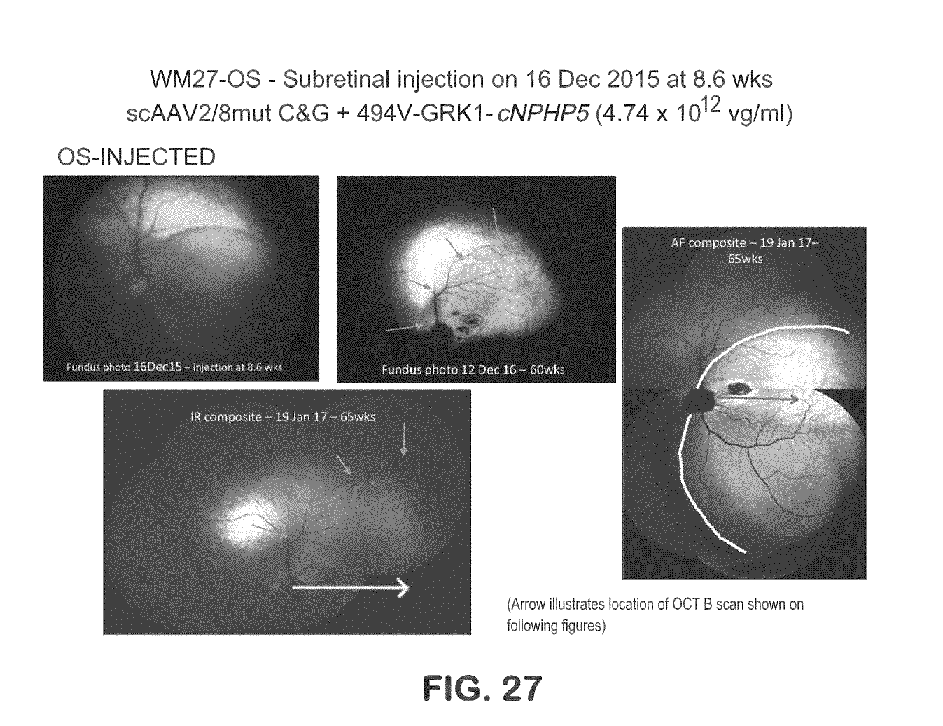

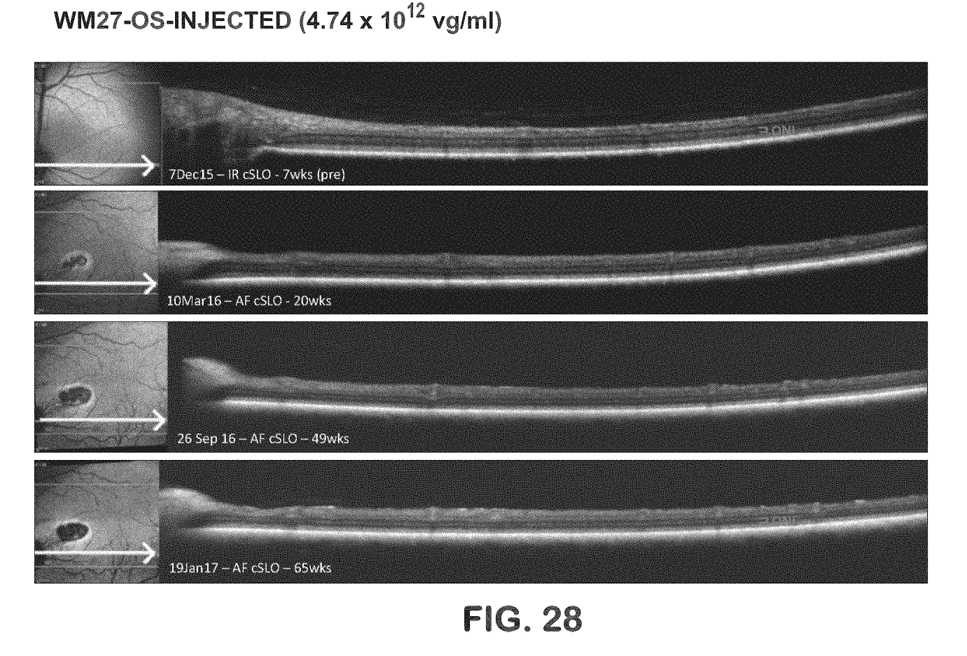

Treatment of two NPHP5 mutant dogs at 8.6 weeks of age after the onset of retinal degeneration by subretinal injection of scAAV2/8mut C&G+T494V-GRK1-cNPHP5 (4.74×10¹² vg/ml titer; 100 μl volume), resulted in remarkable sustained preservation of both rod (FIG. 23) and cone (FIG. 24) ERG function in the treated eyes for over 1 year. Similarly the scAAV2/8 mut C&G+T494V-GRK1-cNPHP5 vector construct at a titer of 4.74×10¹² vg/ml had positive effect on preservation of retinal vasculature and outer nuclear layer (ONL) thickness in the treated area of the injected eye, while ongoing degeneration occurred in surrounding untreated areas as well as in the contralateral uninjected eye (FIGS. 25-28).

These results show that structural and functional rescue of rods and cones can be achieved with the scAAV2/8 mut C&G+T494V-GRK1-cNPHP5 vector construct even when treatment is initiated after the onset of photoreceptor degeneration.

### Example 4

An experiment was designed to further evaluate the canine NPHP5 transgene in the capsid variant scAAV2/8mut C&G+T494V at a later stage of disease when rod and cone structure is severely compromised and ERG function is lost. The scAAV2/8mut C&G+T494V-GRK1-cNPHP5 vector construct was delivered by subretinal injection in an NPHP5 mutant dog after the onset of retinal degeneration (at 13.9 wks).

Animals were treated as follows:

| | | | | Age at | | |
|---|---|---|---|---|---|---|
| Dog | Genotype | Sex | DOB | injection | Right Eye (OD) | Left Eye (OS) |
| AS2- | crd2 A | M | 30 Oct. 2015 | 13.9 wks | Not injected | scAAV2/8mut |
| 408 | | | | | | C&G+T494V− |
| | | | | | | GRK1−cNPHP5 |
| | | | | | | 4.74E+12 vg/ml |
| | | | | | | 150 μl SR |

On date of injection, pupils were dilated (3× at 30 min interval) with Tropicamide/Phenylephrine/Atropine. Subretinal (SR) injection aiming for the Area Centralis was performed under (propofol induction) isoflurane gas anesthesia. The injected viral preparation (˜150 μl) contained the test vector listed in the table above and a small amount of an AAV2/5 carrying the reporter gene GFP to facilitate detection at later time points of the treated area by non-invasive retinal imaging (scanning confocal laser ophthalmoscopy, autofluorescence mode).

Eye exams were performed pre-injection, 24 hrs PI and on a weekly basis for 8 weeks, then monthly. At the following time points assessment of retinal function by electroretinography (ERG) was performed in each eye: at 13.9 weeks of age (pre-injection), and at approx. 20, 28 and 51 wks of age (post-injection). Retinal structure and outer nuclear layer (ONL) thickness was assessed by cSLO/OCT non-invasive retinal imaging in each eye at approx. 13 weeks of age (pre-injection time point) and after injection at approx. 30 and 53 weeks of age.

### Results:

No clinical signs of ocular/retinal toxicity were observed in the eye treated with the vector listed above throughout the in life study duration.

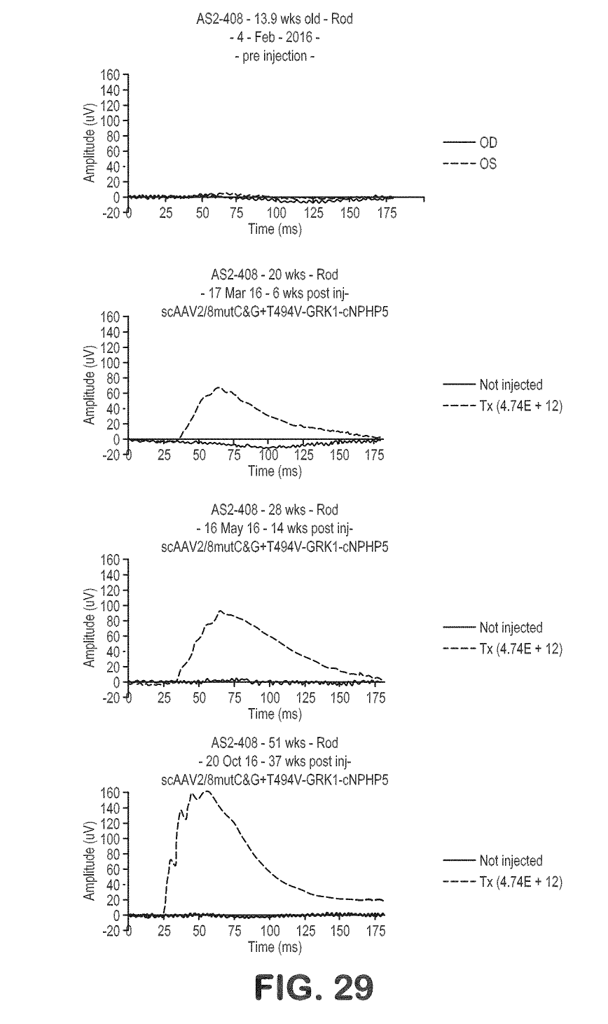

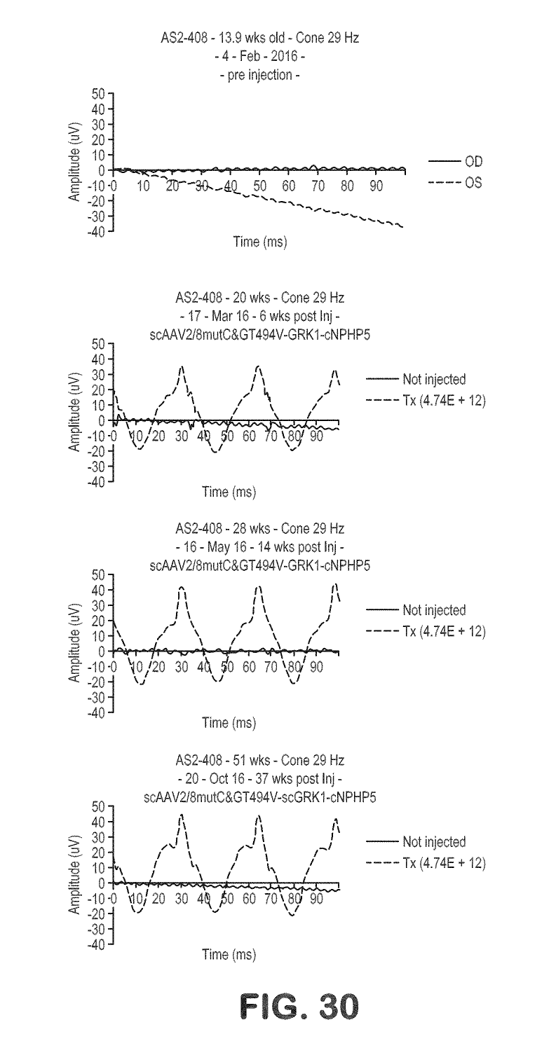

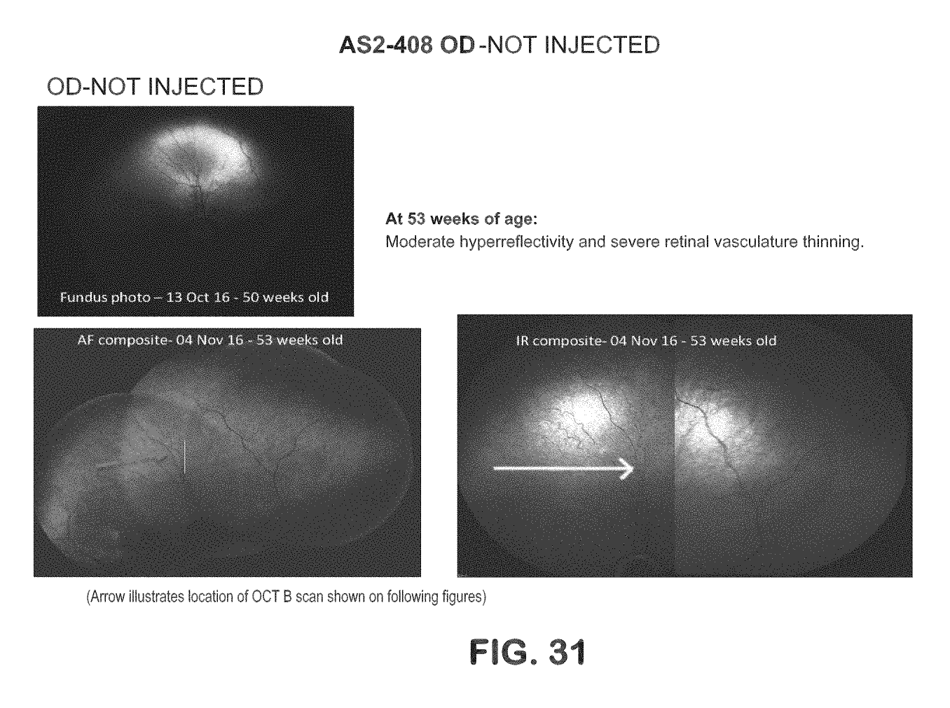

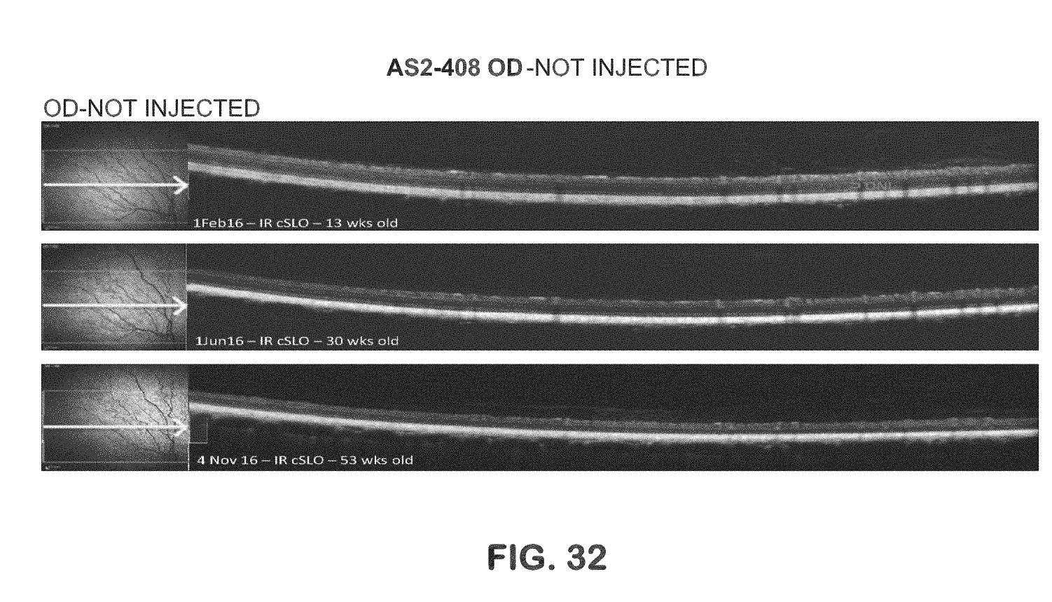

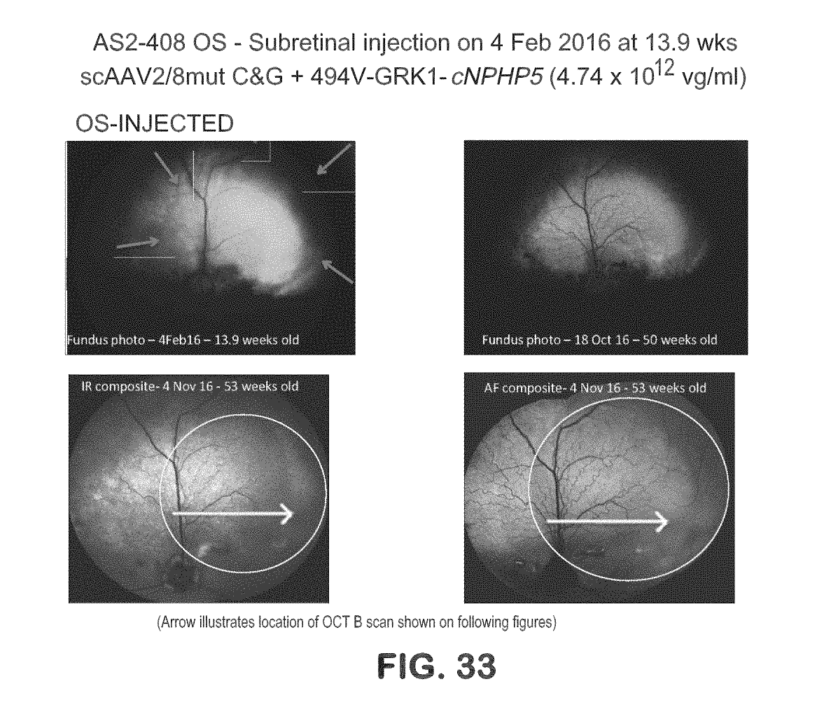

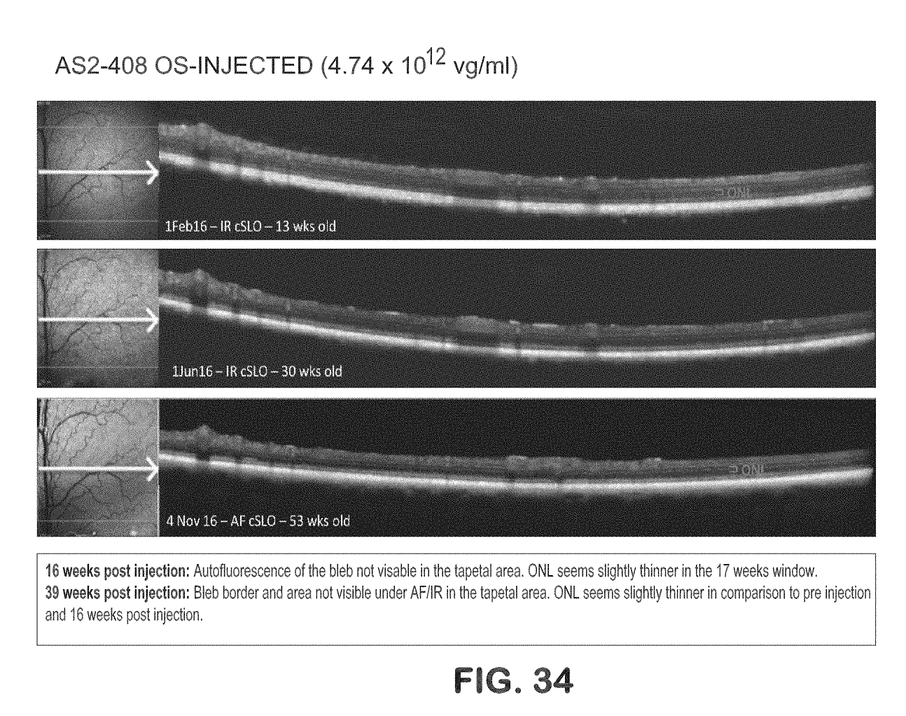

Treatment of an NPHP5 mutant dog at 13.9 weeks of age well after the onset of retinal degeneration by subretinal injection of scAAV2/8mut C&G+T494V-GRK1-cNPHP5 (4.74×10¹² vg/ml titer; 150 μl volume), resulted in remarkable recovery of both rod (FIG. 29) and cone (FIG. 30) ERG function in the treated eye that was absent at 13.9 weeks prior to treatment delivery. The ERG response increased over the course of 37 weeks suggesting a progressive improvement in the retinal rewiring in the treated area. Similarly the scAAV2/8 mut C&G+T494V-GRK1-cNPHP5 vector construct at a titer of 4.74×10¹² vg/ml had positive effect on preservation of retinal vasculature and outer nuclear layer (ONL) thickness in the treated area of the injected eye, while ongoing degeneration occurred in surrounding untreated areas as well as in the contralateral uninjected eye (FIGS. 31-34).

These results show that structural and functional recovery of rods and cones can be achieved with the scAAV2/8 mut C&G+T494V-GRK1-cNPHP5 vector construct even when treatment is initiated at an advanced stage of degeneration with significant photoreceptor death and loss of retinal function.

All patents, patent applications and other references, including U.S. Provisional Patent application No. 62/301,266 and the Sequence Listing cited in this specification, are hereby incorporated by reference in their entirety.

What is claimed is:

1. A recombinant adeno-associated virus (AAV) comprising an AAV capsid protein and a nucleic acid sequence encoding a normal NPHP5 protein, or fragment thereof, under the control of regulatory sequences which express the NPHP5 in the photoreceptor cells of a subject.

2. The rAAV according to claim 1, wherein the rAAV comprises an AAV8 capsid, or variant thereof.

3. The rAAV according to claim 2, wherein the AAV8 capsid variant comprises a tyrosine to phenylalanine mutation.

4. The rAAV according to claim 3, wherein the AAV8 capsid comprises a Y733F mutation.

5. The rAAV according to claim 3, wherein the AAV8 capsid comprises Y447F, Y733F and T494V mutations.

6. The rAAV according to claim 1, wherein the NPHP5 protein is a human sequence.

7. The rAAV according to claim 1, wherein the rAAV comprises an AAV5 capsid, or variant thereof.

8. The rAAV according to claim 1, wherein the NPHP5 protein has the sequence of SEQ ID NO: 1.

9. The rAAV according to claim 8, wherein the NPHP5 protein is encoded by the nucleic acid sequence shown in SEQ ID NO: 3, or a variant thereof.

10. The rAAV according to claim 1, wherein the NPHP5 protein has the sequence of SEQ ID NO: 2.

11. The rAAV according to claim 10, wherein the NPHP5 protein is encoded by the nucleic acid sequence shown in SEQ ID NO: 4, or a variant thereof.

12. The rAAV according to claim 1, wherein the rAAV is a self-complementary AAV.

13. The rAAV according to claim 1, wherein the regulatory sequences comprise a human GRK1 promoter.

14. The rAAV according to claim 1, wherein the regulatory sequences comprise an IRBP promoter.

15. The rAAV according to claim 1, comprising an AAV2/5 capsid protein and a nucleic acid sequence encoding a normal NPHP5 protein under the control of an IRPB promoter.

16. The rAAV according to claim 1, comprising a self-complementary AAV2/8(Y733F) capsid protein and a nucleic acid sequence encoding a normal NPHP5 protein under the control of a GRK1 promoter.

17. The rAAV according to claim 1, comprising a self-complementary AAV2/8(Y447F+733F+T494V) capsid protein and a nucleic acid sequence encoding a normal NPHP5 protein under the control of a GRK1 promoter.

18. A method of preventing, arresting progression of or ameliorating vision loss associated with LCA-ciliopathy in a subject, said method comprising administering to said subject an effective concentration of a composition comprising a recombinant adeno-associated virus (AAV) carrying a nucleic acid sequence encoding a normal NPHP5 protein, or fragment thereof, under the control of regulatory sequences which express the NPHP5 in the photoreceptor cells of said subject, and a pharmaceutically acceptable carrier.

19. The method according to claim 18, wherein the composition is administered by subretinal injection.

20. A method of treating or preventing LCA-ciliopathy in a subject in need thereof comprising:
(a) identifying subject having, or at risk of developing, LCA-ciliopathy;
(b) performing genotypic analysis and identifying a mutation in the NPHP5 gene;
(c) performing non-invasive retinal imaging and functional studies and identifying areas of retained photoreceptors that could be targeted for therapy;
(d) administering to said subject an effective concentration of a composition comprising a recombinant virus carrying a nucleic acid sequence encoding a normal photoreceptor cell-specific gene under the control of a promoter sequence which expresses the product of said gene in said photoreceptor cells, and a pharmaceutically acceptable carrier,
wherein said LCA-ciliopathy is prevented, arrested or ameliorated.


SEQUENCE LISTING (14 sequences)

SEQ ID NO: 1 - protein, 598 aa, Homo sapiens
MKPTGTDPRILSIAAEVAKSPEQNVPVILLKLKEIINITPLGSSELKKIKQDIYCYDLIQYCLLVLSQDYSRIQGGWTTISQLTQILSHCCVGLEPGEDAEEFYNELLPSAAENFLVLGRQLQTCFINAAKAEEKDELLHFFQIVTDSLFWLLGGHVELIQNVLQSDHFLHLLQADNVQIGSAVMMMLQNILQINSGDLLRIGRKALYSILDEVIFKLFSTPSPVIRSTATKLLLLMAESHQEILILLRQSTCYKGLRRLLSKQETGTEFSQELRQLVGLLSPMVYQEVEEQKLHQAACLIQAYWKGFQTRKRLKKLPSAVIALQRSFRSKRSKMLLEINRQKEEEDLKLQLQLQRQRAMRLSRELQLSMLEIVHPGQVEKHYREMEEKSALNIQKHWRGYRERKNFHQQRQSLIEYKAAVTLQRAALKFLAKCRKKKKLFAPWRGLQELTDARRVELKKRVDDYVRRHLGSPMSDVVSRELHAQAQERLQHYFMGRALEERAQQHREALIAQISTNVEQLMKAPSLKEAEGKEPELFLSRSRPVAAKAKQAHLTTLKHIQAPWWKKLGEESGDEIDVPKDELSIELENLFIGGTKPP

SEQ ID NO: 2 - protein, 598 aa, Canis familiaris
MKPTGTDPRILSLAAEVAKSPEQNVPVILLKLKEIINNTPLGSSELKKIKQDIYCYDLIQYCLLVLSQDCSRIQGGWTTISQLTQILSHCCVGLEPGEDAEEFYNELLPSAAENFLVLGRRLQTCFINSAKGEEKDELLHSFQIVTDSLFWLLGGHVQLIQNVLQSDHFLHLLQTDNVQIGSTVMTMLQNILQINSGDLLRIEGKILHSILDEVVFKLLSTPNPVIRSTATKLLLLMTESHQEILILLRLSACYKGLRSLLNKHEPGTEFSQELGQLIALLTPKVYQEVEDQKLHQAACLIQAYWKGFQTRKRLKKLPSAVITLQRSFRSKRTKILLKLNKQKEEEDRRLQLQLQRQRAMRLSRELRLSMLEIVHPGQVEKYNREIEEKSALIIQKHWRGYRERKNFRQQRPSLTEYKAAVILQRATLKFLAKCRKKKKLFAPWRGLQDLTDARRVELKQQVDDYLRRHPSSQMSDMTSRELHSQAQEQLQHYLMGRALEERAQQHREALMAQISTNIEQLMKAPSLKEAEGKEPELFLSRSRPVAAKAKQAHLTALKHIQAPWWKKLGEEAGDEIDVPKDEFSLELGTLFIGGTKPP

SEQ ID NO: 3 - DNA, 1797 bp, Homo sapiens
atgaagccaacaggtacagacccaaggatcttatctatagctgctgaagttgcaaaaagccctgagcagaatgtccctgttatactgttgaagttaaaagaaataataaacatcacacctttaggaagctcagagttgaagaaaatcaaacaagatatatattgttatgatctcattcaatattgcctcttggtcctcagtcaagattattctcgaatccagggtggttggactacaatttcccagcttacacagatattaagccattgctgtgtgggcttggagccaggagaagatgcagaggaattttacaatgaattacttccatcagctgcagaaaattttctagttttggggagacaattacaaacatgttttatcaatgcagctaaggctgaagaaaaagatgaattactacactttttccaaattgtgactgattctctcttctggcttttgggaggccatgttgaacttattcagaatgtactacaaagtgatcatttcttacatttactgcaagctgacaatgtccaaataggatctgcagtcatgatgatgctacagaatatattacagatcaacagtggtgatttactcagaataggaagaaaagccctgtattcaattttagatgaagttattttcaagcttttttcaactcctagtccagttataagaagtactgctacaaaactcctactgttgatggctgaatcccatcaggaaattttgattttactgagacaaagtacctgctacaaaggactcagacgtctactaagtaaacaggaaactgggactgaattcagtcaagaacttagacagcttgttggccttttaagcccaatggtctatcaggaagtagaagagcagaaactacatcaagcagcatgcttgattcaagcctattggaagggttttcagacaagaaagagattaaagaagcttccatctgctgtgattgctttgcagaggagtttcagatccaaacgatcaaagatgttgctggagataaataggcagaaggaagaagaggacctcaaattacaattgcaacttcaaagacagagagccatgagactttcccgagaattgcagctgagtatgctcgaaatagttcatccaggtcaggtggagaaacactatcgggaaatggaagagaaatcagcactgaatatccagaaacattggagagggtacagggaaaggaaaaattttcaccaacagaggcagtctctcatagagtataaagcagctgtcacacttcaaagagcagcgcttaaattcctagcgaagtgccgtaagaaaaagaaactatttgctccttggcgaggactccaagaactcactgatgcacgccgagttgaactgaagaaacgagtggatgactatgtcagaagacatttgggctctccaatgtcagatgtggtcagtagggagctccatgcccaagctcaagaacgactgcaacactactttatgggcagggccctagaagagcgagcccagcagcacagagaagctctgatagcacagatcagcaccaacgttgaacagctaatgaaggcaccaagtctgaaggaggcagaagggaaagaacctgagctcttcctaagtagatccaggcctgtggcagccaaggccaagcaggcccatctcacaaccctgaagcacatacaagcaccctggtggaagaagcttggagaagaatctggagatgagattgatgttccaaaggatgagcttagtatagaattagaaaatttattcattggtggaaccaaaccaccttag

SEQ ID NO: 4 - DNA, 1797 bp, Canis familiaris
atgaagccaacaggtacagacccaaggatcttatctctagctgctgaagttgcaaaaagtcctgagcaaaatgtccctgttatactattgaagttaaaagaaataataaacaacacacctttaggaagctcagagttgaagaaaatcaaacaagatatatattgttatgacctcattcagtattgccttttggtgctcagtcaagattgttctcgaatccagggaggttggactacaatatcccaacttacacagatattaagccactgctgtgtgggcttggagccaggagaagatgcagaggaattttacaatgaattactcccatcagctgcagaaaattttctggttttggggagacgattgcaaacatgtttcatcaattcagctaagggtgaagaaaaagatgaattactacactcgttccaaattgtgaccgattctctcttctggctcttaggaggccatgttcaactcatccaaaatgtactacaaagtgatcatttcttgcacttactgcaaactgacaatgttcaaataggatctacagtcatgactatgctacagaacatactacagatcaacagtggtgatttactcagaatagaaggaaaaatcctacattcaattttagatgaagttgttttcaagcttttatcaactcctaacccagtcataagaagtactgctacaaagctcctactgctgatgactgaatcccatcaggaaattttgattttactgagactaagtgcctgctacaaaggactcagaagtctattaaacaaacatgagcctgggacagagtttagtcaagaacttggacagcttattgcccttttaacccctaaggtctatcaggaagtagaagatcagaaactacatcaagcagcttgcttgattcaagcttattggaagggtttccaaactagaaaaagattaaagaagcttccatctgctgtgattactttgcagaggagtttcagatctaaacgaaccaagatattactaaagctaaataagcagaaagaagaagaggaccgcagattacagttgcaacttcaaagacagagagccatgagattgtcccgagaattacggctgagtatgctcgaaatagttcatccaggtcaggtggaaaaatataatcgggaaatagaagagaaatcagccttgattatccagaaacactggagagggtacagggaaaggaaaaattttcgccaacagaggccatctctcacggaatataaagcagctgtcatacttcaaagagcaactcttaaattcctagcaaagtgccgtaagaaaaagaaactatttgctccttggcgaggacttcaagatctcaccgatgcacggagagttgaattaaagcaacaagtggatgactatctcagaagacatccgagctctcaaatgtcagatatgactagcagagagctccattcccaagctcaagaacaactgcaacactaccttatgggcagggccctagaagagagagcccagcagcacagggaggctctgatggctcagatcagcaccaacattgaacagttaatgaaggcaccgagtctgaaggaggcagaagggaaagaacctgaactcttcctaagtagatccaggcctgtggcagctaaggccaagcaggcccatcttactgccctgaagcatatacaggcaccttggtggaagaagcttggggaagaagcaggagatgagattgatgttccaaaggatgagtttagtttagaattaggaactttattcattggtggaaccaaacccccttag

SEQ ID NO: 5 - DNA, 292 bp, Homo sapiens
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggc

SEQ ID NO: 6 - DNA, 235 bp, Homo sapiens
agcacagtgtctggcatgtagcaggaactaaaataatggcagtgattaatgttatgatatgcagacacaacacagcaagataagatgcaatgtaccttctgggtcaaaccaccctggccactcctccccgatacccagggttgatgtgcttgaattagacaggattaaaggcttactggagctggaagccttgccccaactcaggagtttagccccagaccttctgtccaccagc

SEQ ID NO: 7 - DNA, 5650 bp, artificial
  constructed sequence
ctgcaggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccagcacagtgtctggcatgtagcaggaactaaaataatggcagtgattaatgttatgatatgcagacacaacacagcaagataagatgcaatgtaccttctgggtcaaaccaccctggccactcctccccgatacccagggttgatgtgcttgaattagacaggattaaaggcttactggagctggaagccttgccccaactcaggagtttagccccagaccttctgtccaccagcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaaacggtttgtacccgcggccgccaccatgaagccaacaggtacagacccaaggatcttatctctagctgctgaagttgcaaaaagtcctgagcaaaatgtccctgttatactattgaagttaaaagaaataataaacaacacacctttaggaagctcagagttgaagaaaatcaaacaagatatatattgttatgacctcattcagtattgccttttggtgctcagtcaagattgttctcgaatccagggaggttggactacaatatcccaacttacacagatattaagccactgctgtgtgggcttggagccaggagaagatgcagaggaattttacaatgaattactcccatcagctgcagaaaattttctggttttggggagacgattgcaaacatgtttcatcaattcagctaagggtgaagaaaaagatgaattactacactcgttccaaattgtgaccgattctctcttctggctcttaggaggccatgttcaactcatccaaaatgtactacaaagtgatcatttcttgcacttactgcaaactgacaatgttcaaataggatctacagtcatgactatgctacagaacatactacagatcaacagtggtgatttactcagaatagaaggaaaaatcctacattcaattttagatgaagttgttttcaagcttttatcaactcctaacccagtcataagaagtactgctacaaagctcctactgctgatgactgaatcccatcaggaaattttgattttactgagactaagtgcctgctacaaaggactcagaagtctattaaacaaacatgagcctgggacagagtttagtcaagaacttggacagcttattgcccttttaacccctaaggtctatcaggaagtagaagatcagaaactacatcaagcagcttgcttgattcaagcttattggaagggtttccaaactagaaaaagattaaagaagcttccatctgctgtgattactttgcagaggagtttcagatctaaacgaaccaagatattactaaagctaaataagcagaaagaagaagaggaccgcagattacagttgcaacttcaaagacagagagccatgagattgtcccgagaattacggctgagtatgctcgaaatagttcatccaggtcaggtggaaaaatataatcgggaaatagaagagaaatcagccttgattatccagaaacactggagagggtacagggaaaggaaaaattttcgccaacagaggccatctctcacggaatataaagcagctgtcatacttcaaagagcaactcttaaattcctagcaaagtgccgtaagaaaaagaaactatttgctccttggcgaggacttcaagatctcaccgatgcacggagagttgaattaaagcaacaagtggatgactatctcagaagacatccgagctctcaaatgtcagatatgactagcagagagctccattcccaagctcaagaacaactgcaacactaccttatgggcagggccctagaagagagagcccagcagcacagggaggctctgatggctcagatcagcaccaacattgaacagttaatgaaggcaccgagtctgaaggaggcagaagggaaagaacctgaactcttcctaagtagatccaggcctgtggcagctaaggccaagcaggcccatcttactgccctgaagcatatacaggcaccttggtggaagaagcttggggaagaagcaggagatgagattgatgttccaaaggatgagtttagtttagaattaggaactttattcattggtggaaccaaacccccttaggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccagg

SEQ ID NO: 8 - DNA, 6467 bp, artificial
  constructed sequence
cgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctcgatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgaagccaacaggtacagacccaaggatcttatctctagctgctgaagttgcaaaaagtcctgagcaaaatgtccctgttatactattgaagttaaaagaaataataaacaacacacctttaggaagctcagagttgaagaaaatcaaacaagatatatattgttatgacctcattcagtattgccttttggtgctcagtcaagattgttctcgaatccagggaggttggactacaatatcccaacttacacagatattaagccactgctgtgtgggcttggagccaggagaagatgcagaggaattttacaatgaattactcccatcagctgcagaaaattttctggttttggggagacgattgcaaacatgtttcatcaattcagctaagggtgaagaaaaagatgaattactacactcgttccaaattgtgaccgattctctcttctggctcttaggaggccatgttcaactcatccaaaatgtactacaaagtgatcatttcttgcacttactgcaaactgacaatgttcaaataggatctacagtcatgactatgctacagaacatactacagatcaacagtggtgatttactcagaatagaaggaaaaatcctacattcaattttagatgaagttgttttcaagcttttatcaactcctaacccagtcataagaagtactgctacaaagctcctactgctgatgactgaatcccatcaggaaattttgattttactgagactaagtgcctgctacaaaggactcagaagtctattaaacaaacatgagcctgggacagagtttagtcaagaacttggacagcttattgcccttttaacccctaaggtctatcaggaagtagaagatcagaaactacatcaagcagcttgcttgattcaagcttattggaagggtttccaaactagaaaaagattaaagaagcttccatctgctgtgattactttgcagaggagtttcagatctaaacgaaccaagatattactaaagctaaataagcagaaagaagaagaggaccgcagattacagttgcaacttcaaagacagagagccatgagattgtcccgagaattacggctgagtatgctcgaaatagttcatccaggtcaggtggaaaaatataatcgggaaatagaagagaaatcagccttgattatccagaaacactggagagggtacagggaaaggaaaaattttcgccaacagaggccatctctcacggaatataaagcagctgtcatacttcaaagagcaactcttaaattcctagcaaagtgccgtaagaaaaagaaactatttgctccttggcgaggacttcaagatctcaccgatgcacggagagttgaattaaagcaacaagtggatgactatctcagaagacatccgagctctcaaatgtcagatatgactagcagagagctccattcccaagctcaagaacaactgcaacactaccttatgggcagggccctagaagagagagcccagcagcacagggaggctctgatggctcagatcagcaccaacattgaacagttaatgaaggcaccgagtctgaaggaggcagaagggaaagaacctgaactcttcctaagtagatccaggcctgtggcagctaaggccaagcaggcccatcttactgccctgaagcatatacaggcaccttggtggaagaagcttggggaagaagcaggagatgagattgatgttccaaaggatgagtttagtttagaattaggaactttattcattggtggaaccaaacccccttaggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggaaccccactccctctctgcgcgctcgctcgctcactgaggccgggcgaccaaaggtcgcccgacgcccgggctttgcccgggcggcctcagtgagcgagcgagcgcgcagctgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaatccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctggaaatccattcgccattcaggctgcgcaactgttgggaagggcgatcggtgcgggcctcttcgctattacgccagctggcg

SEQ ID NO: 9 - DNA, 7397 bp, Artificial Sequence
  constructed sequence
ctagtaacggccgccagtgtgctggaattcggctttatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggtgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtacttcttgtctagaactcaaacaacaggaggcacggcaaatacgcagactctgggcttcagccaaggtgggcctaatacaatggccaatcaggcaaagaactggctgccaggaccctgttaccgccaacaacgcgtcagtactgtaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacgcgtttcctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgctttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcggatcga

SEQ ID NO: 10 - protein, 738 aa, Artificial Sequence
  constructed sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTVTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 11 - DNA, 2802 bp, Artificial Sequence
  constructed sequence
ctgcaggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccagcacagtgtctggcatgtagcaggaactaaaataatggcagtgattaatgttatgatatgcagacacaacacagcaagataagatgcaatgtaccttctgggtcaaaccaccctggccactcctccccgatacccagggttgatgtgcttgaattagacaggattaaaggcttactggagctggaagccttgccccaactcaggagtttagccccagaccttctgtccaccagcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaaacggtttgtacccgcggccgccaccatgaagccaacaggtacagacccaaggatcttatctctagctgctgaagttgcaaaaagtcctgagcaaaatgtccctgttatactattgaagttaaaagaaataataaacaacacacctttaggaagctcagagttgaagaaaatcaaacaagatatatattgttatgacctcattcagtattgccttttggtgctcagtcaagattgttctcgaatccagggaggttggactacaatatcccaacttacacagatattaagccactgctgtgtgggcttggagccaggagaagatgcagaggaattttacaatgaattactcccatcagctgcagaaaattttctggttttggggagacgattgcaaacatgtttcatcaattcagctaagggtgaagaaaaagatgaattactacactcgttccaaattgtgaccgattctctcttctggctcttaggaggccatgttcaactcatccaaaatgtactacaaagtgatcatttcttgcacttactgcaaactgacaatgttcaaataggatctacagtcatgactatgctacagaacatactacagatcaacagtggtgatttactcagaatagaaggaaaaatcctacattcaattttagatgaagttgttttcaagcttttatcaactcctaacccagtcataagaagtactgctacaaagctcctactgctgatgactgaatcccatcaggaaattttgattttactgagactaagtgcctgctacaaaggactcagaagtctattaaacaaacatgagcctgggacagagtttagtcaagaacttggacagcttattgcccttttaacccctaaggtctatcaggaagtagaagatcagaaactacatcaagcagcttgcttgattcaagcttattggaagggtttccaaactagaaaaagattaaagaagcttccatctgctgtgattactttgcagaggagtttcagatctaaacgaaccaagatattactaaagctaaataagcagaaagaagaagaggaccgcagattacagttgcaacttcaaagacagagagccatgagattgtcccgagaattacggctgagtatgctcgaaatagttcatccaggtcaggtggaaaaatataatcgggaaatagaagagaaatcagccttgattatccagaaacactggagagggtacagggaaaggaaaaattttcgccaacagaggccatctctcacggaatataaagcagctgtcatacttcaaagagcaactcttaaattcctagcaaagtgccgtaagaaaaagaaactatttgctccttggcgaggacttcaagatctcaccgatgcacggagagttgaattaaagcaacaagtggatgactatctcagaagacatccgagctctcaaatgtcagatatgactagcagagagctccattcccaagctcaagaacaactgcaacactaccttatgggcagggccctagaagagagagcccagcagcacagggaggctctgatggctcagatcagcaccaacattgaacagttaatgaaggcaccgagtctgaaggaggcagaagggaaagaacctgaactcttcctaagtagatccaggcctgtggcagctaaggccaagcaggcccatcttactgccctgaagcatatacaggcaccttggtggaagaagcttggggaagaagcaggagatgagattgatgttccaaaggatgagtttagtttagaattaggaactttattcattggtggaaccaaacccccttaggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 12 - DNA, 2817 bp, Artificial Sequence
  constructed sequence
cgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctcgatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgaagccaacaggtacagacccaaggatcttatctctagctgctgaagttgcaaaaagtcctgagcaaaatgtccctgttatactattgaagttaaaagaaataataaacaacacacctttaggaagctcagagttgaagaaaatcaaacaagatatatattgttatgacctcattcagtattgccttttggtgctcagtcaagattgttctcgaatccagggaggttggactacaatatcccaacttacacagatattaagccactgctgtgtgggcttggagccaggagaagatgcagaggaattttacaatgaattactcccatcagctgcagaaaattttctggttttggggagacgattgcaaacatgtttcatcaattcagctaagggtgaagaaaaagatgaattactacactcgttccaaattgtgaccgattctctcttctggctcttaggaggccatgttcaactcatccaaaatgtactacaaagtgatcatttcttgcacttactgcaaactgacaatgttcaaataggatctacagtcatgactatgctacagaacatactacagatcaacagtggtgatttactcagaatagaaggaaaaatcctacattcaattttagatgaagttgttttcaagcttttatcaactcctaacccagtcataagaagtactgctacaaagctcctactgctgatgactgaatcccatcaggaaattttgattttactgagactaagtgcctgctacaaaggactcagaagtctattaaacaaacatgagcctgggacagagtttagtcaagaacttggacagcttattgcccttttaacccctaaggtctatcaggaagtagaagatcagaaactacatcaagcagcttgcttgattcaagcttattggaagggtttccaaactagaaaaagattaaagaagcttccatctgctgtgattactttgcagaggagtttcagatctaaacgaaccaagatattactaaagctaaataagcagaaagaagaagaggaccgcagattacagttgcaacttcaaagacagagagccatgagattgtcccgagaattacggctgagtatgctcgaaatagttcatccaggtcaggtggaaaaatataatcgggaaatagaagagaaatcagccttgattatccagaaacactggagagggtacagggaaaggaaaaattttcgccaacagaggccatctctcacggaatataaagcagctgtcatacttcaaagagcaactcttaaattcctagcaaagtgccgtaagaaaaagaaactatttgctccttggcgaggacttcaagatctcaccgatgcacggagagttgaattaaagcaacaagtggatgactatctcagaagacatccgagctctcaaatgtcagatatgactagcagagagctccattcccaagctcaagaacaactgcaacactaccttatgggcagggccctagaagagagagcccagcagcacagggaggctctgatggctcagatcagcaccaacattgaacagttaatgaaggcaccgagtctgaaggaggcagaagggaaagaacctgaactcttcctaagtagatccaggcctgtggcagctaaggccaagcaggcccatcttactgccctgaagcatatacaggcaccttggtggaagaagcttggggaagaagcaggagatgagattgatgttccaaaggatgagtttagtttagaattaggaactttattcattggtggaaccaaacccccttaggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggaaccccactccctctctgcgcgctcgctcgctcactgaggccgggcgaccaaaggtcgcccgacgcccgggctttgcccgggcggcctcagtgagcgagcgagcg

SEQ ID NO: 13 - DNA, 2802 bp, Artificial Sequence
  constructed sequence
ctgcaggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccagcacagtgtctggcatgtagcaggaactaaaataatggcagtgattaatgttatgatatgcagacacaacacagcaagataagatgcaatgtaccttctgggtcaaaccaccctggccactcctccccgatacccagggttgatgtgcttgaattagacaggattaaaggcttactggagctggaagccttgccccaactcaggagtttagccccagaccttctgtccaccagcctcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaaacggtttgtacccgcggccgccaccatgaagccaacaggtacagacccaaggatcttatctatagctgctgaagttgcaaaaagccctgagcagaatgtccctgttatactgttgaagttaaaagaaataataaacatcacacctttaggaagctcagagttgaagaaaatcaaacaagatatatattgttatgatctcattcaatattgcctcttggtcctcagtcaagattattctcgaatccagggtggttggactacaatttcccagcttacacagatattaagccattgctgtgtgggcttggagccaggagaagatgcagaggaattttacaatgaattacttccatcagctgcagaaaattttctagttttggggagacaattacaaacatgttttatcaatgcagctaaggctgaagaaaaagatgaattactacactttttccaaattgtgactgattctctcttctggcttttgggaggccatgttgaacttattcagaatgtactacaaagtgatcatttcttacatttactgcaagctgacaatgtccaaataggatctgcagtcatgatgatgctacagaatatattacagatcaacagtggtgatttactcagaataggaagaaaagccctgtattcaattttagatgaagttattttcaagcttttttcaactcctagtccagttataagaagtactgctacaaaactcctactgttgatggctgaatcccatcaggaaattttgattttactgagacaaagtacctgctacaaaggactcagacgtctactaagtaaacaggaaactgggactgaattcagtcaagaacttagacagcttgttggccttttaagcccaatggtctatcaggaagtagaagagcagaaactacatcaagcagcatgcttgattcaagcctattggaagggttttcagacaagaaagagattaaagaagcttccatctgctgtgattgctttgcagaggagtttcagatccaaacgatcaaagatgttgctggagataaataggcagaaggaagaagaggacctcaaattacaattgcaacttcaaagacagagagccatgagactttcccgagaattgcagctgagtatgctcgaaatagttcatccaggtcaggtggagaaacactatcgggaaatggaagagaaatcagcactgaatatccagaaacattggagagggtacagggaaaggaaaaattttcaccaacagaggcagtctctcatagagtataaagcagctgtcacacttcaaagagcagcgcttaaattcctagcgaagtgccgtaagaaaaagaaactatttgctccttggcgaggactccaagaactcactgatgcacgccgagttgaactgaagaaacgagtggatgactatgtcagaagacatttgggctctccaatgtcagatgtggtcagtagggagctccatgcccaagctcaagaacgactgcaacactactttatgggcagggccctagaagagcgagcccagcagcacagagaagctctgatagcacagatcagcaccaacgttgaacagctaatgaaggcaccaagtctgaaggaggcagaagggaaagaacctgagctcttcctaagtagatccaggcctgtggcagccaaggccaagcaggcccatctcacaaccctgaagcacatacaagcaccctggtggaagaagcttggagaagaatctggagatgagattgatgttccaaaggatgagcttagtatagaattagaaaatttattcattggtggaaccaaaccaccttaggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 14 - DNA, 2817 bp, Artificial Sequence
  constructed sequence
cgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctcgatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgaagccaacaggtacagacccaaggatcttatctatagctgctgaagttgcaaaaagccctgagcagaatgtccctgttatactgttgaagttaaaagaaataataaacatcacacctttaggaagctcagagttgaagaaaatcaaacaagatatatattgttatgatctcattcaatattgcctcttggtcctcagtcaagattattctcgaatccagggtggttggactacaatttcccagcttacacagatattaagccattgctgtgtgggcttggagccaggagaagatgcagaggaattttacaatgaattacttccatcagctgcagaaaattttctagttttggggagacaattacaaacatgttttatcaatgcagctaaggctgaagaaaaagatgaattactacactttttccaaattgtgactgattctctcttctggcttttgggaggccatgttgaacttattcagaatgtactacaaagtgatcatttcttacatttactgcaagctgacaatgtccaaataggatctgcagtcatgatgatgctacagaatatattacagatcaacagtggtgatttactcagaataggaagaaaagccctgtattcaattttagatgaagttattttcaagcttttttcaactcctagtccagttataagaagtactgctacaaaactcctactgttgatggctgaatcccatcaggaaattttgattttactgagacaaagtacctgctacaaaggactcagacgtctactaagtaaacaggaaactgggactgaattcagtcaagaacttagacagcttgttggccttttaagcccaatggtctatcaggaagtagaagagcagaaactacatcaagcagcatgcttgattcaagcctattggaagggttttcagacaagaaagagattaaagaagcttccatctgctgtgattgctttgcagaggagtttcagatccaaacgatcaaagatgttgctggagataaataggcagaaggaagaagaggacctcaaattacaattgcaacttcaaagacagagagccatgagactttcccgagaattgcagctgagtatgctcgaaatagttcatccaggtcaggtggagaaacactatcgggaaatggaagagaaatcagcactgaatatccagaaacattggagagggtacagggaaaggaaaaattttcaccaacagaggcagtctctcatagagtataaagcagctgtcacacttcaaagagcagcgcttaaattcctagcgaagtgccgtaagaaaaagaaactatttgctccttggcgaggactccaagaactcactgatgcacgccgagttgaactgaagaaacgagtggatgactatgtcagaagacatttgggctctccaatgtcagatgtggtcagtagggagctccatgcccaagctcaagaacgactgcaacactactttatgggcagggccctagaagagcgagcccagcagcacagagaagctctgatagcacagatcagcaccaacgttgaacagctaatgaaggcaccaagtctgaaggaggcagaagggaaagaacctgagctcttcctaagtagatccaggcctgtggcagccaaggccaagcaggcccatctcacaaccctgaagcacatacaagcaccctggtggaagaagcttggagaagaatctggagatgagattgatgttccaaaggatgagcttagtatagaattagaaaatttattcattggtggaaccaaaccaccttaggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggaaccccactccctctctgcgcgctcgctcgctcactgaggccgggcgaccaaaggtcgcccgacgcccgggctttgcccgggcggcctcagtgagcgagcgagcg